(19) United States
(12) Patent Application Publication
(10) Pub. No.: US 2016/0032310 A1
(43) Pub. Date: Feb. 4, 2016

(54) METHODS AND COMPOSITIONS FOR OBTAINING USEFUL EPIGENETIC TRAITS

(71) Applicant: Michael E. Fromm, Lincoln, NE (US)

(72) Inventor: Michael E. Fromm, Lincoln, NE (US)

(21) Appl. No.: 14/716,681

(22) Filed: May 19, 2015

Related U.S. Application Data
(60) Provisional application No. 62/000,756, filed on May 20, 2014.

(51) Int. Cl. C12N 15/82; A01H 1/02; C12N 9/10

(52) U.S. Cl. CPC C12N 15/8261; C12N 9/1007; A01H 1/02; C12Y 201/01

(57) ABSTRACT

The present invention provides methods for obtaining plants that exhibit useful traits by expression of a recombinant DNA methyltransferase in progenitor plants. Methods for identifying genetic loci that provide for useful traits in plants and plants produced with those loci are also provided. In addition, plants that exhibit the useful traits, parts of the plants including seeds, and products of the plants are provided as well as methods of using the plants. Recombinant DNA vectors and transgenic plants comprising those vectors that express a recombinant DNA methyltransferase are also provided.

### CROSS-REFERENCE TO RELATED APPLICATIONS

This application claims the benefit of U.S. Provisional Patent Application No. 62/000,756 filed May 20, 2014, which is incorporated herein by reference in its entirety.

### INCORPORATION OF SEQUENCE LISTING

The sequence listing contained in the file named “NontargetedMethylases_ST25.txt”, which is 169,664 bytes in size (measured in operating system MS-Windows), contains 31 sequences, and is contemporaneously filed with this specification by electronic submission (using the United States Patent Office EFS-Web filing system) and is incorporated herein by reference in its entirety. The information recorded in computer readable form is identical to the written sequence listing submitted in the provisional patent application 62/000,756 filed on May 20, 2014, and the computer readable submission of sequences includes no new matter.

### STATEMENT REGARDING FEDERALLY SPONSORED RESEARCH OR DEVELOPMENT

None.

### BACKGROUND OF INVENTION

Plant genomes contain relatively large amounts of 5-methylcytosine (5meC; Kumar et al. 2013 J Genet 92(3): 629-666). Other than silencing transposable elements and repeated sequences, the biological roles of 5meC are still emerging. Intercrossing a low methylation mutant plant with a normally methylated plant resulted in heritable changes in DNA methylation in the plant genome that affected some plant phenotypic traits (Cortijo et al. 2014 Science. 2014 Mar. 7; 343(6175):1145-8). Over expression of Arabidopsis MET1, a DNA methyltransferase predominantly responsible for CG maintenance methylation, in Arabidopsis resulted in plants that flowered earlier (U.S. Pat. Nos. 6,011,200 and 6,444,469). This method focused specifically on MET1 type of DNA methyltransferases, which predominantly use CG as their DNA methylation substrate. U.S. Pat. No. 5,750,868 describes the use of a bacterial DAM methyltransferase to cause male sterility in plants by methylation at the A position of a GATC sequence.

Earlier studies of DNA methylation changes in Arabidopsis suggest amenability of the epigenome to recurrent selection and also suggest that it is feasible to establish new and stable epigenetic states (F. Johannes et al. PLoS Genet. 5, e1000530 (2009); F. Roux et al. Genetics 188, 1015 (2011); Cortijo et al., Science. 2014 Mar. 7; 343(6175):1145-8). Manipulation of the Arabidopsis met1 and ddmt mutants has allowed the creation of epi-RIL populations that show both heritability of novel methylation patterning and epiallelic segregation, underscoring the likely influence of epigenomic variation in plant adaptation (F. Roux et al. Genetics 188, 1015 (2011); Cortijo et al., Science. 2014 Mar. 7; 343(6175):1145-8). In natural populations, a large proportion of the epiallelic variation detected in Arabidopsis is found as CpG methylation within gene-rich regions of the genome (C. Becker et al. Nature 480, 245 (2011), R. J. Schmitz et al. Science 334, 369 (2011). Induction of traits that exhibit cytoplasmic inheritance (Redei Mutat. Res. 18, 149-162, 1973; Sandhu et al. Proc Natl Acad Sci USA. 104:1766-70, 2007) or that exhibit nuclear inheritance by suppression of the MSH1 gene has also been reported (WO 2012/151254; Xu et al. Plant Physiol. Vol. 159:711-720, 2012).

### Plant Transformation Methods.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton). Plant transformation methods for producing transgenic plants include, but are not limited to methods for: Alfalfa as described in U.S. Pat. No. 7,521,600; Canola and rapeseed as described in U.S. Pat. No. 5,750,871; Cotton as described in U.S. Pat. No. 5,846,797; corn as described in U.S. Pat. No. 7,682,829. Indica rice as described in U.S. Pat. No. 6,329,571; Japonica rice as described in U.S. Pat. No. 5,591,616; wheat as described in U.S. Pat. No. 8,212,109; barley as described in U.S. Pat. No. 6,100,447; potato as described in U.S. Pat. No. 7,250,554; sugar beet as described in U.S. Pat. No. 6,531,649; and, soybean as described in U.S. Pat. No. 8,592,212. Many additional methods or modified methods for plant transformation are known to those skilled in the art for many plant species.

### SUMMARY OF INVENTION

In general, this invention generates useful epigenetic changes in the progeny of one or more plants or plant cells subjected to expression of a recombinant DNA methyltransferase, whether propagated via seeds or vegetatively, to produce plants with improved useful traits such as increased yield and/or tolerance to stress or disease. In general, the methods and compositions described herein provide useful and/or alternative methods to increase yields and useful traits in plants derived from progenitor plants or plant cells with increased DNA methylation due to expression of one or more recombinant DNA methyltransferases.

Methods for producing a plant useful for plant breeding, methods for identifying one or more altered chromosomal loci in a plant that are useful for plant breeding, methods for obtaining plants comprising modified chromosomal loci that are useful for plant breeding, improved plants from said plant breeding, parts of those plants including cells, leafs, stems, flowers and seeds, methods of using the plants and plant parts, and products of those plants and plant parts, including processed products such as a feed or a meal are provided herein.

Methods for producing a plant exhibiting a useful trait comprising the steps of: (a) expressing a recombinant DNA methyltransferase in a plant or plant cell; and, (b) selecting one or more progeny plants derived from the plant or plant cell of step (a) that exhibit(s) a useful trait, thereby producing a plant exhibiting a useful trait are provided herein. In certain embodiments the parent plant of step (a) or at least a portion of the progeny plants of step (b) exhibit one or more MSH1-dr phenotypes. In certain embodiments the recombinant DNA methyltransferase is a member of the DRM2 group of DNA methyltransferases. In certain embodiments the recombinant DNA methyltransferase is a member of the CMT3 group of DNA methyltransferases. In certain embodiments the progeny of step (b) do not contain the recombinant DNA methyltransferase of step (a). In certain embodiments the progeny of step (b) are outcrossed and then selfed, and do not contain the recombinant DNA methyltransferase of step (a). In certain embodiments the plant comprises a recombinant DNA methyltransferase. In certain embodiments the plant comprising a recombinant DNA methyltransferase selected from the group consisting of DRM2 group of DNA methyltransferases or CMT3 group of DNA methyltransferases. In certain embodiments the progeny comprise said recombinant DNA methyltransferase or lack said recombinant DNA methyltransferase. In certain embodiments the progeny of step (b) are derived from a parental plant by an outcross to produce F1 seeds and subsequent selfing to produce F2 seeds. In certain embodiments expression is effected with a transgene comprising a promoter that is selectively expressed in cells containing sensory plastids and that is operably linked to a DNA methyltransferase. In certain embodiments the promoter is a MSH1 promoter, PPD3 promoter, PSBO1 promoter, or PSBO2 promoter. In certain embodiments expression is effected with a transgene comprising an inducible promoter that is operably linked to a DNA methyltransferase. In certain embodiments expression of a DNA methyltransferase is effected with an operably linked viral vector. In certain embodiments the methylation status of one or more genes of in the nuclear genome of the progeny of step (b) are monitored. In certain embodiments the methylation status of a pericentromeric region of a chromosome is monitored. In certain embodiments a first and/or later generation progeny plant of step (b) exhibits one or more regions of pericentromeric CHG and/or CHH hypermethylation in comparison to a control plant not comprising a recombinant DNA methyltransferase. In certain embodiments the method further comprises the step of producing seed from: i) a selfed progeny plant or plants; ii) an out-crossed progeny plant or plants; or, iii) both of an out-crossed and selfed progeny plant or plants. In certain embodiments the method further comprises the step of producing seed from: (i) a selfed progeny plant or plants selected in step (b); or from (ii) an outcrossed progeny plant or plants selected in step (b).

In certain embodiments the method comprises: (i) outcrossing or selfing the first parental plant or progeny thereof to obtain an F1 generation of plants, wherein the first parental plant or progeny thereof exhibits one or more Msh1-dr phenotypes; (ii) screening and selecting a population of plants obtained from the outcross for the presence of the useful trait and the absence of Msh1-dr phenotypes; (iii) obtaining seed from the selected population of step (ii) or, optionally, repeating steps (ii) and (iii) on a population of plants grown from the seed obtained from the selected population. In certain embodiments the useful trait is selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to expression of a recombinant DNA methyltransferase. In certain embodiments the useful trait is associated with one or more epigenetic changes in one or more chromosomal regions. In certain embodiments the selected progeny plant(s) or progeny thereof exhibit an improvement in the trait in comparison to a plant that had not been subjected to the recombinant DNA methyltransferase but was otherwise isogenic to the first parental plant or plant cell. In certain embodiments the plant is a crop plant. In certain embodiments the crop plant is selected from the group consisting of corn, soybean, cotton, wheat, rice, tomato, tobacco, millet, potato, sorghum, alfalfa, sunflower, canola, peanut, canola (Brassica napus, Brassica rapa ssp.), coffee (Coffea spp.), coconut (Cocos nucijra), pineapple (Ananas comosus), citrus trees (Citrus spp.), cocoa (Theobroma cacao), poplar, sugar beets (Beta vulgaris), sugarcane (Saccharum spp.), oats, barley, vegetables, ornamentals, and conifers.

In certain embodiments a plant or population of plants produced by any of the aforementioned methods, wherein the plant or population of plants exhibits an improvement in at least one useful trait in comparison to a plant that had not been subjected to the recombinant DNA methyltransferase but was otherwise isogenic to the first parental plant or plant cell and wherein the plant or at least 25%-50%, 50%-70%, 70%-80%, 80%-90%, 90%-95%, or 95%-100% of the population of plants exhibit the trait. In certain embodiments the plant is inbred. In certain embodiments the seed or a plant obtained therefrom exhibits the improvement in at least one useful trait. In certain embodiments a processed product from the plant or population of plants of or from the seed thereof, wherein the product comprises a detectable amount of a nuclear chromosomal DNA comprising one or more epigenetic changes that were induced by the recombinant DNA methyltransferase. In certain embodiments the product is oil, meal, lint, bulls, or a pressed cake. Methods for producing a seed lot, comprising the steps of selfing a population of plants of any of the aforementioned methods, and harvesting a seed lot therefrom, wherein at least about 25%-50%, 50%-70%, 70%-80%, 80%-90%, 90%-95%, or 95%-100% of harvested seed or plants obtained therefrom exhibit the improvement in at least one useful trait are also provided.

Methods for identifying one or more altered chromosomal loci in a plant that can confer a useful trait comprising the steps of: (a) comparing DNA methylation status of one or more nuclear chromosomal regions in a reference plant that does not exhibit the useful trait to one or more corresponding nuclear chromosomal regions in a test plant that does exhibit the useful trait, wherein the test plant was obtained by the method of any of the aforementioned methods; and, (b) selecting for one or more altered nuclear chromosomal loci present in the test plant with a DNA methylation status that is distinct from the DNA methylation status in the reference plant, wherein the selected chromosomal loci are associated with the useful trait are provided herein. In certain embodiments the DNA methylation status comprises CG hypermethylation and/or CHG and/or CHH hypermethylation. In certain embodiments the selection comprises isolating a plant or progeny plant comprising the altered chromosomal locus or obtaining a nucleic acid associated with the altered chromosomal locus. In certain embodiments the reference plant and the test plant are both obtained from a population of progeny plants obtained from a parental plant or plant cell subjected to expression of a recombinant DNA methyltransferase. In certain embodiments both the reference plant and the parental plant or plant cell were isogenic prior to expression of a recombinant DNA methyltransferase in the parental plant or plant cell. In certain embodiments of the aforementioned methods the useful trait is selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to expression of a recombinant DNA methyltransferase. In certain embodiments of the aforementioned methods the plant comprises an altered chromosomal locus.

Methods for producing a plant exhibiting a useful trait comprising the steps of: (a) introducing a nuclear chromosomal modification associated with a useful trait into a plant, wherein the chromosomal modification comprises an epigenetic change induced by anyone of the aforementioned methods that is associated with the useful trait; and, (b) selecting for a plant or plants that comprise the nuclear chromosomal modification and exhibit the useful trait are provided herein. In certain embodiments the method further comprises the step of producing seed from: i) a selfed progeny plant of the selected plant or plants of step (b), ii) an out-crossed progeny plant of the selected plant or plants of step (b), or, iii) from both of a selfed and an outcrossed progeny plant of the selected plant or plants of step (b). In certain embodiments the chromosomal modification comprises CHG hypermethylation and/or CHH hypermethylation. In certain embodiments the chromosomal modification comprises a chromosomal mutation and the plant is selected by assaying for the presence of the the chromosomal mutation. In certain embodiments the plant is selected by assaying for the presence of the useful trait.

Methods for producing a plant having a useful trait comprising the steps of: (a) crossing a first plant to-a second plant wherein said first plant or its progenitor plant or plant cell is subjected to expression of a recombinant DNA methyltransferase; and, (b) selecting one or more progeny plants having a useful trait, thereby producing a plant exhibiting a useful trait are provided herein. In certain embodiments the first plant or its progenitor plant do not exhibit any MSH1-dr phenotypes. In certain embodiments the first plant or its progenitor plant exhibit MSH1-dr phenotypes. In certain embodiments the second plant or its progenitor plant are not subjected to expression of a recombinant DNA methyltransferase. In certain embodiments the second plant or its progenitor plant are subjected to expression of a recombinant DNA methyltransferase or to suppression of MSH 1, In certain embodiments the first plant(s) of step (a) exhibits an improvement in a useful trait in comparison to a control plant. In certain embodiments of any of the aforementioned methods about 1% to about 45% of the population of progeny plants are selected for the useful trait in step (b).

Methods for identifying one or more altered chromosomal loci that is useful for plant breeding comprising the steps of: (a) comparing one or more chromosomal regions in a reference plant to one or more corresponding chromosomal regions in a test plant that exhibits a useful trait, wherein said test plant was obtained from a parental plant or plant cell subjected to expression of a recombinant DNA methyltransferase; and, (b) selecting for one or more altered chromosomal loci present in said test plant that are less altered in said reference plant and that are associated with said useful trait are provided herein. In certain embodiments the test plant of step (a) does not exhibit any MSH1-dr phenotype and/or has enhanced growth relative to a control plant.

Methods for producing a plant that is useful for plant breeding comprising the steps of: (a) introducing a chromosomal modification associated with a useful trait into a plant, wherein said chromosomal modification is induced in a parental plant or plant, cell subjected to expression of a recombinant DNA methyltransferase; and, (b) selecting for a plant that comprises said chromosomal modification, thereby producing a plant that is useful for plant breeding are provided herein. In certain embodiments the plant of step (a) does not exhibit any MSH1-dr phenotype and/or has enhanced growth relative to a control plant.

Methods for producing a plant having a useful trait comprising the steps of: (a) selling a plant wherein said plant or its progenitor is subjected to expression of a recombinant DNA methyltransferase, wherein said plant or its progenitor plant does not exhibit any MSH1-dr phenotypes; and, (b) selecting one or more progeny plants having a useful trait, thereby producing a plant exhibiting a useful trait are provided herein.

Methods of identifying a plant harboring a useful trait or that is useful for plant breeding comprising the steps of: (a) crossing a candidate plant to a second plant, wherein said candidate plant or its progenitor is subjected to expression of a recombinant DNA methyltransferase, wherein said candidate plant does not exhibit a Msh1-dr phenotype; and, (b) identifying one or more progeny plants from the cross of step (a) that exhibit a useful trait or that is useful for plant breeding to a greater extent than the candidate plant, the second plant, or a control plant, thereby identifying the candidate plant as a plant that harbors a useful trait or that is useful for plant breeding are also provided. In certain embodiments the control plant is progeny of a cross between; (i) a plant that is not progeny of a selfed plant, a crossed plant, or parent thereof that is or had been subjected to expression of a recombinant DNA methyltransferase; and (ii) a plant that is isogenic to the second plant. In certain embodiments a plant, progeny thereof, or seed thereof that harbors a useful trait, wherein said plant, progeny thereof, or seed thereof is identified or identifiable by the aforementioned methods.

Methods for producing a plant exhibiting new combinations of altered chromosomal loci useful for breeding comprising the steps of: (a) crossing a plant comprising altered chromosomal loci induced by expression of a recombinant DNA methyltransferase in said plant or its progenitor to produce progeny; and, (b) assaying the DNA methylation of said progeny to identify and select individuals with new combinations of altered chromosomal loci, thereby producing a plant exhibiting new combinations of altered chromosomal loci useful for breeding are provided herein. In certain embodiments one or more altered chromosomal loci are selected from the group consisting of pericentromeric regions, CG enhanced genes, CG depleted genes, transposable elements, transposable elements containing genes, and transposable elements in pericentromeric regions. In certain embodiments the DNA methylation of one or more altered chromosomal loci occurs at CHG or CHH sites within a DNA region selected from the group consisting of pericentromeric regions, transposable elements, transposable elements containing genes, and transposable elements in pericentromeric regions. In certain embodiments the DNA methylation of one or more altered chromosomal loci occurs at CG sequences near or within CG altered genes.

Methods for producing a plant exhibiting new combinations of altered chromosomal loci useful for breeding comprising the steps of: (a) crossing a plant comprising altered chromosomal loci induced expression of a recombinant DNA methyltransferase in said plant or its progenitor to produce progeny; and, (b) assaying one or more sRNAs of said progeny to identify and select individuals with new combinations of altered chromosomal loci, thereby producing a plant exhibiting new combinations of altered chromosomal loci useful for breeding are provided herein. In certain embodiments one or more sRNAs assayed have sequence homology to one or more regions selected from the group consisting of pericentromeric regions, CG enhanced genes, CG depleted genes, transposable elements, transposable elements containing genes, and transposable elements in pericentromeric regions.

Methods for identifying a plant with altered chromosomal loci useful for plant breeding comprising the steps of: (a) assaying DNA methylation of one or more plants comprising altered chromosomal loci induced by expression of a recombinant DNA methyltransferase in said plants or their progenitor(s); and, (b) identifying one or more plants from step (a) comprising one or more altered chromosomal loci selected from the group consisting of pericentromeric regions, CG enhanced genes, CG depleted genes, transposable elements, transposable elements containing genes, and transposable elements in pericentromeric regions, thereby identifying a plant with altered chromosomal loci useful for plant breeding are provided herein. In certain embodiments the DNA methylation of one or more altered chromosomal loci occurs at CHG or CHH at DNA sequences selected from the group consisting of pericentromeric regions, transposable elements, transposable elements containing genes, and transposable elements in pericentromeric regions. In certain embodiments the DNA methylation of one or more altered chromosomal loci occurs at CG sequences near or within CG altered genes.

Methods for identifying a plant with altered chromosomal loci useful for plant breeding comprising the steps of: (a) assaying one or more sRNAs of one or more plants comprising altered chromosomal loci induced by expression of a recombinant DNA methyltransferase in said plants or their progenitor(s); and, (b) identifying one or more plants from step (a) comprising increases or decreases in one or more sRNAs with homology at DNA sequences to one or more regions selected from the group of altered chromosomal loci consisting of pericentromeric regions, CG enhanced genes, CG depleted genes, transposable elements, transposable elements containing genes, and transposable elements in pericentromeric regions, thereby identifying a plant with altered chromosomal loci useful for plant breeding are provided herein.

Methods for producing a plant exhibiting new combinations of altered chromosomal loci useful for breeding comprising the steps of: (a) selfing a plant comprising altered chromosomal loci induced by expression of a recombinant DNA methyltransferase in said plant or its progenitor to produce progeny; and, (b) assaying the DNA methylation at altered chromosomal loci of said progeny to identify and select individuals with new combinations of altered chromosomal loci are provided herein. In certain embodiments one or more altered chromosomal loci are selected from the group consisting of pericentromeric regions, CG enhanced genes, CG depleted genes, transposable elements, transposable elements containing genes, and transposable elements in pericentromeric regions. In certain embodiments the DNA methylation of one or more altered chromosomal loci occurs at one or more CHG or CHH sites within one or more DNA regions selected from the group consisting of pericentromeric regions, transposable elements, transposable elements containing genes, and transposable elements in pericentromeric regions. In certain embodiments the DNA methylation of one or more altered chromosomal loci occurs at one or more CG sequences near or within one or more CG altered genes.

Methods for producing a plant exhibiting new combinations of altered chromosomal loci useful for breeding comprising the steps of: (a) selfing a plant comprising altered chromosomal loci induced expression of a recombinant DNA methyltransferase in said plant or its progenitor to produce progeny; and, (b) assaying one or more sRNAs of said progeny to identify and select individuals with new combinations of altered chromosomal loci. In certain embodiments one or more sRNAs assayed have sequence homology to one or more regions selected from the group of altered chromosomal loci consisting of pericentromeric regions, CG enhanced genes, CG depleted genes, transposable elements, transposable elements containing genes, and transposable elements in pericentromeric regions.

Methods for selecting a plant comprising one or more altered chromosomal loci useful for plant breeding comprising the steps of: (a) comparing the DNA methylation status of one or more nuclear chromosomal regions in a reference plant to one or more corresponding nuclear chromosomal regions in a candidate plant, wherein said candidate plant or its progenitor plant or cell was obtained by expression of a recombinant DNA methyltransferase; and, (b) selecting a candidate plant comprising one or more nuclear chromosomal regions present in the candidate plant with a DNA methylation status that is distinct from the DNA methylation status in the reference plant, thereby selecting a plant comprising one or more altered chromosomal loci useful for plant breeding are provided herein.

Methods for selecting a plant comprising one or more altered chromosomal loci useful for plant breeding comprising the steps of: (a) comparing one or more sRNAs with homology to one or more nuclear chromosomal regions in a reference plant to one or more sRNAs from corresponding nuclear chromosomal regions in a candidate plant, wherein said candidate plant or its progenitor plant or cell was obtained by expression of a recombinant DNA methyltransferase; and, (b) selecting a candidate plant comprising one or more sRNA with abundances or sequences that are distinct from the sRNAs in the reference plant, thereby selecting a plant comprising one or more altered chromosomal loci useful for plant breeding are also provided herein.

In certain embodiments a recombinant DNA construct comprising a promoter that is selectively expressed in cells containing sensory plastids and that is operably linked to a sequence for expression of a recombinant DNA methyltransferase is provided herein. In certain embodiments the promoter is selected from the group consisting of Msh1, PPD3, or PSBO1, or PSBO2 promoters. In certain embodiments the sequence for expression of a recombinant DNA methyltransferase is selected from the group of genes consisting of the DRM2 group of DNA methyltransferases or CMT3 group of DNA methyltransferases. In certain embodiments a recombinant DNA construct expressing a member selected from the group consisting of DRM2 group of DNA methyltransferases or CMT3 group of DNA methyltransferases is provided herein. In certain embodiments a recombinant DNA comprising a constitutive promoter that expresses a recombinant DNA methyltransferase is provided herein. In certain embodiments a recombinant DNA comprising an inducible promoter that expresses a recombinant DNA methyltransferase is provided. In certain embodiments a transgenic plant or plant cell comprising the aforementioned recombinant DNA constructs is provided herein.

Methods for producing a seed lot comprising the steps of: (a) selecting a first sub-population of plants exhibiting a useful trait associated with an epigenetic change at one or more nuclear chromosomal loci from a first population of plants derived from one or more progenitor plants subjected to expression of a recombinant DNA methyltransferase; and, (b) obtaining a seed lot from the first selected sub-population of step (a) or, optionally, repeating step (a) on a second population of plants grown from the seed obtained from the first selected sub-population of plants are provided herein. In certain embodiments the epigenetic change was induced by expression of a recombinant DNA methyltransferase selected from the DRM2 group or CMT3 group of DNA methyltransferases. In certain embodiments the epigenetic change is associated with CG hypermethylation and/or CHG and/or CHH hyper-methylation at one or more nuclear chromosomal loci in comparison to a control plant that does not exhibit the useful trait. In certain embodiments a plurality of plants in the first sub-population exhibit heritable pericentromeric CHG and/or CHH hyper-methylation. In certain embodiments at least 25%-50%, 50%-70%, 70%-80%, 80%-90%, 90%-95%, or 95%-100% of progeny plants grown from the seed lot obtained in step (b) exhibit the useful trait associated with an epigenetic change. In certain embodiments the seed or progeny plants grown from the seed comprise inbred and/or hybrid germplasm that is epigenetically heterogenous. In certain embodiments seed lot is produced by any of the aforementioned methods.

Also provided is a seed lot comprising seed wherein at least 25%-50%, 50%-70%, 70%-80%, 80%-90%, 90%-95%, or 95%-100% of progeny plants grown from the seed exhibit a useful trait associated with one or more epigenetic changes produced by any of aforementioned methods, wherein the epigenetic changes are associated with CG hyper-methylation and/or CHG and/or CHH hyper-methylation at one or more nuclear chromosomal loci in comparison to a control plant that does not exhibit the useful trait. In certain embodiments the useful trait is selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that lacks the epigenetic change(s). In certain embodiments the seed or progeny plants grown from the seed comprise inbred and/or hybrid germplasm that is epigenetically heterogenous.

Also provided by any of the aforementioned methods is a useful trait selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to expression of a recombinant DNA methyltransferase.

Also provided is a plant made by any of the aforementioned methods. In certain embodiments the plant is from the group consisting of corn, wheat, rice, sorghum, millet, tomatoes, potatoes, soybeans, tobacco, cotton, canola, alfalfa, rapeseed, sugar beets, sugarcane, a vegetable, a fruit, a bush, or a tree.

Also provided is a clonal propagate derived from a plant, plant part, seed, or plant cell of any of the aforementioned methods. In certain embodiments the plant is grafted to a scion or rootstock from the plant of any the aforementioned methods. In certain embodiments the progeny of the graft are provided.

Methods of any of the aforementioned methods wherein the recombinant DNA methyltransferase gene encodes a protein comprising at least 25%-50%, 50%-70%, 70%-80%, 80%-90%, 90%-95%, or 95%-100% amino acid homology in the catalytic domain to a member selected from the DRM2 group or CMT3 group of DNA methyltransferases are also provided.

Also provided are a plant or progeny or vegetative propagule thereof that exhibits a useful trait or that is useful for plant breeding that is made by any of the aforementioned methods.

Methods of identifying a plant harboring a useful trait comprising the steps of: (a) crossing a candidate plant exhibiting one or more Msh1-dr phenotypes, wherein said plant or its progenitor was subjected to expression of a recombinant DNA methyltransferase, to a second plant to produce F1 progeny; (b) identifying one or more F1 progeny or later generation progeny plants from step (a) that exhibit a useful trait to a greater extent than the candidate plant, the second plant, or a control plant, thereby identifying the candidate plant as a plant that harbors a useful trait are provided herein. In certain embodiments the second plant of step (a) is isogenic or does not display heterosis when crossed with a control plant of the same genotype as the candidate plant of step (a). In certain embodiments the control plant of step (b) is derived from a cross of a plant of the candidate plant's parental genotype of step (a) that lacks any Msh1-dr phenotypes to a plant of the same genotype as the second plant of step (a).

Methods of identifying a plant harboring a useful trait comprising the steps of: (a) crossing a candidate plant exhibiting one or more Msh1-dr phenotypes, wherein said plant or its progenitor was subjected to expression of a recombinant DNA methyltransferase, to a second plant to produce F1 progeny; (b) selfing said F1 progeny of step (a) to produce progeny; and, (c) identifying one or more progeny plants from step (b) that exhibit a useful trait to a greater extent than the candidate plant, the second plant, or a control plant, thereby identifying the candidate plant as a plant that harbors a useful trait or that is useful for plant breeding are provided herein. In certain embodiments the second plant of step (a) is isogenic or does not display heterosis when crossed with a control plant of the same genotype as the candidate plant of step (a). In certain embodiments the control plant of step (c) is derived from a cross of a plant of the candidate plant's parental genotype of step (a) that lacks any Msh1-dr phenotypes to a plant of the same genotype as the second plant of step (a).

Methods of identifying a plant harboring a useful trait comprising the steps of: (a) crossing a candidate plant not exhibiting one or more Msh1-dr phenotypes, wherein said plant or its progenitor was subjected to expression of a recombinant DNA methyltransferase, to a second plant to produce F1 progeny; (b) identifying one or more F1 progeny or later generation progeny plants from step (a) that exhibit a useful trait to a greater extent than the candidate plant, the second plant, or a control plant, thereby identifying the candidate plant as a plant that harbors a useful trait are provided herein. In certain embodiments the second plant of step (a) is isogenic or does not display heterosis when crossed with a control plant of the same genotype as the candidate plant of step (a).

### BRIEF DESCRIPTION OF THE DRAWINGS

The accompanying drawings, which are incorporated in and form a part of the specification, illustrate certain embodiments of the present invention. In the drawings:

FIG. 1. A Clustal Omega alignment of the catalytic domains of 18 DRM2 plant proteins is shown. The Genbank protein ID number is shown at the left of each row, and as each protein is longer than a single row, this ID is shown in subsequent blocks of 18 rows each along the length of the proteins. The degree of amino acid relatedness at a specific alignment position (. or : for similar amino acids) or identity in all 18 proteins (* for identical amino acids) is shown at the bottom of each row.

FIG. 2. A Clustal Omega alignment of the catalytic domains of 16 CMT3 plant proteins is shown. The Genbank protein ID number is shown at the left of each row, and as each protein is longer than a single row, this ID is shown in subsequent blocks of 16 rows each along the length of the proteins. The degree of amino acid relatedness at a specific alignment position (. or : for similar amino acids) or identity in all 16 proteins (* for identical amino acids) is shown at the bottom of each row.

### DETAILED DESCRIPTION

### Definitions

As used herein, the phrases “altered chromosomal loci” (used as singular or plural herein) or “altered chromosomal locus” (singular) refer to regions of a chromosome that have undergone a heritable and reversible epigenetic change due to expression of a recombinant DNA methyltransferase relative to the corresponding parental chromosomal loci prior to expression of a recombinant DNA methyltransferase. The altered chromosomal loci can occur in any of the generations of progeny derived from a progenitor plant subjected to expression of a recombinant DNA methyltransferase. Heritable and reversible epigenetic changes in altered chromosomal loci include, but are not limited to, methylation of chromosomal DNA, and in particular, methylation of cytosine residues to 5-methylcytosine residues. As used herein, “chromosomal loci” refer to loci in chromosomes located in the nucleus of a cell. Altered chromosomal loci can be assayed for DNA methylation or sRNA derived from these regions. Altered chromosomal loci have altered DNA methylation levels, and/or altered levels of sRNA derived from these regions, relative to the corresponding parental chromosomal loci prior to expression of a recombinant DNA methyltransferase or to a parental chromosome in a lineage not subjected to expression of a recombinant DNA methyltransferase.

As used herein, the terms “assaying” or “assayed” refer to methods for determining the amounts, or sequences, or both, of DNA methylation or sRNA, corresponding to one or more nuclear chromosomal regions for DNA or with homology to one or more nuclear chromosomal regions for sRNA. The nuclear chromosomal regions assayed for DNA methylation can be a single nucleotide position or a region greater than this. Preferably the DNA methylation is from a region comprising one or more CG, CHG, or CHH1 sites and is compared to the corresponding parental chromosomal loci prior to MSH1 suppression. sRNA can be measured for a single type of sRNA, one or more sRNAs, or a whole population of sRNAs by methods known to those skilled in the art.

As used herein, the phrases “CG altered gene” or “CG altered genes” refer to a gene or genes with increased or decreased levels of DNA methylation (5meC) at CG nucleotides within or near a gene or genes. The region near a gene is within 5,000 bp, preferably within 1,000 bp, of either the 5′ or 3′ end of the gene or genes.

As used herein, the phrase “CG enhanced genes” refers to genes identified as altered chromosomal loci with higher levels of DNA methylation derived from a chromosomal region relative to the comparable chromosomal region of a reference plant.

As used herein, the terms “CG depleted genes” refers to genes identified as altered chromosomal loci with lower levels of DNA methylation derived from a chromosomal region relative to the comparable chromosomal region of a reference plant.

As used herein, the phrase “chromosomal modification” refers to any of: a) an “altered chromosomal loci” and an “altered chromosomal locus”; b) “mutated chromosomal loci”, a “mutated chromosomal locus”, “chromosomal mutations” and a “chromosomal mutation”; or c) a transgene.

As used herein, the phrases “clonal propagate” or “vegetatively propagated” refer to a plant or progeny thereof obtained from a plant, plant cell, tissue culture, or tissue, or seed that is propagated as a plant cutting or tuber cutting or tuber or tissue culture process such as embryogenesis or organogenesis. Clonal propagates can be obtained by methods including but not limited to regenerating whole plants from plant cells, plant embryos, cuttings, tubers, and the like. Various techniques used for such clonal propagation include, but are not limited to, meristem culture, somatic embryogenesis, thin cell layer cultures, adventitious shoot culture, and callus culture.

As used herein, the phrases “commercially synthesized” or “commercially available” DNA refer to the availability of any sequence of 15 bp up to 1000 bp in length or longer from DNA synthesis companies that provide a DNA sample containing the sequence submitted to them.

As used herein, the term “comprising” means “including but not limited to”.

As used herein the phrase “Conservatively modified variants” includes individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

As used herein, the phrase “crop plant” includes, but is not limited to, cereal, seed, grain, fruit, ornamental, and vegetable plants.

As used herein the phrase “conserved amino acids in the catalytic domain” refers to the conserved amino acids that are identical in the DRM2 group of DNA methyltransferases in Example 17 or in the CMT3 group of DNA methyltransferases in Example 18.

As used herein the phrases “CMT3” or “CMT3 group” refer to DNA methyltransferases of the DNMT1 general family (Xu et al., Curr Med Chem. 2010; 17(33): 4052-4071; Law and Jacobsen, Nat Rev Genet. 2010 March; 11(3): 204-220; Grace and Bestor Annu. Rev. Biochem. 2005. 74:481-514). Additionally, CMT3 has conserved amino acids in the catalytic domain of CMT3 that are described in Example 18. Proteins comprising DNA methyltransferase activity on CHG and/or CHH DNA sites and that comprise protein regions of at least 25% to 50%, 50% to 60%, 60% to 70%, 70% to 80%, 80% to 90%, 90% to 100%, or 100% identical to the conserved (identical) amino acids identified in Example 18 are members of the CMT3 group of DNA methyltransferases.

As used herein, the phrase “developmental reprograming or term “dr” refers to MSH1-dr phenotypes.

As used herein, the term “discrete variation” or “V_{D}” refers to distinct, heritable phenotypic variation, that includes traits of male sterility, dwarfing, variegation, and/or delayed flowering time that can be observed either in any combination or in isolation.

As used herein the phrases “DRM2” or “DRM2 group” refer to DNA methyltransferases of the DNMT3a/DNMT3b general family (Xu et al., Curr Med Chem. 2010; 17(33): 4052-4071; Law and Jacobsen, Nat Rev Genet. 2010 March; 11(3): 204-220; Grace and Bestor Annu. Rev. Biochem. 2005.74:481-514). Additionally, DRM2 has conserved amino acids in the catalytic domain of DRM2 that are described in Example 17. Proteins comprising DNA methyltransferase activity on CHG and/or CHH DNA sites and that comprise protein regions of at least 25% to 50%, 50% to 60%, 60% to 70%, 70% to 80%, 80% to 90%, 90% to 100%, or 100% identical to the conserved amino acids identified in Example 17 are members of the DMR2 group of DNA methyltransferases.

As used herein, the phrases “epigenetic modifications” or “epigenetic modification” refer to heritable and reversible epigenetic changes that include, but are not limited to, methylation of chromosomal DNA, and in particular, methylation of cytosine residues to 5-methylcytosine residues. Changes in DNA methylation of a region are often associated with changes in sRNA levels with homology to the methylated region.

As used herein, the term “F1” refers to the first progeny of two genetically or epigenetically different plants. “F2” refers to progeny from the self pollination of the F1 plant. “F3” refers to progeny from the self pollination of the F2 plant. “F4” refers to progeny from the self pollination of the F3 plant. “F5” refers to progeny from the self pollination of the F4 plant. “Fn” refers to progeny from the self pollination of the F(n−1) plant, where “n” is the number of generations starting from the initial F1 cross. Crossing to an isogenic line (backcrossing) or unrelated line (outcrossing) at any generation will also use the “Fn” notation, where “n” is the number of generations starting from the initial F1 cross.

As used herein, the phrases “genetically homogeneous” or “genetically homozygous” refer to the two parental genomes provided to a progeny plant as being essentially identical at the DNA sequence level.

As used herein, the phrases “genetically heterogeneous” or “genetically heterozygous” refers to the two parental genomes provided to a progeny plant as being substantially different at the sequence level. That is, one or more genes from the male and female gametes occur in different allelic forms with DNA sequence differences between them.

As used herein, the term “isogenic” refers to the two plants that have essentially identical genomes at the DNA sequence levels level.

As used herein, the phrase “heterotic group” refers to genetically related germplasm that produce superior hybrids when crossed to genetically distinct germplasm of another heterotic group.

As used herein, the phrase “heterologous sequence”, when used in the context of an operably linked promoter, refers to any sequence or any arrangement of a sequence that is distinct from the sequence or arrangement of the sequence with the promoter as it is found in nature. For example, an MSH1 promoter can be operably linked to a heterologous sequence that includes, but is not limited to, recombinant DNA methyltransferase sequences.

“Homology” as used herein refers to sequence similarity between a reference sequence and at least a fragment of a second sequence. Homologs may be identified by any method known in the art, preferably, by using the BLAST or CLUSTAL Omega tool to compare a reference sequence or sequences to a single second sequence or fragment of a sequence or to a database of sequences. As described below, BLAST or CLUSTAL Omega will compare sequences based upon percent identity and similarity.

The terms “identical” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity or percent identity exists over a region that is at least about 50 nucleotides (or 10 amino acids) in length, or more preferably over a region that is 100 to 500 or 1000 or more nucleotides (or 20, 50, 200, or more amino acids) in length. Two examples of algorithms that are suitable for determining percent sequence identity and sequence similarity are the BLAST and BLAST 2.0 algorithms, which are described in Altschul et al. (1997) Nucleic Acids Res 25(17)3389-3402 and Altschul et al. (1990) J. Mol Biol 215(3)-403-410, respectively. The BLASTN program (for nucleotide sequences) or BLASTP program (for amino acid sequences) or CLUSTAL Omega are suitable for most alignments.

As used herein, the phrases “increased DNA methylation” or “decreased DNA methylation” refer to nucleotides, regions, genes, chromosomes, and genomes located in the nucleus that have undergone a change in 5meC (5-methyl cytosine) levels in a plant or progeny plant relative to the corresponding parental chromosomal loci prior to expression of a recombinant DNA methyltransferase.

As used herein, the phrase “loss of function” refers to a diminished, partial, or complete loss of function.

As used herein the terms “microRNA” or “miRNA” refers to both a miRNA that is substantially similar to a native miRNA that occurs in a plant as well as to an artificial miRNA. In certain embodiments, a transgene can be used to produce either a miRNA that is substantially similar to a native miRNA that occurs in a plant or an artificial miRNA.

As used herein, the phrases “MSH1-dr” or “MSH1-dr phenotypes” refers to one or more phenotypes that include leaf variegation, cytoplasmic male sterility (CMS), a reduced growth-rate phenotype, delayed or non-flowering phenotype, leaf wrinkling, increased plant tillering, decreased height, decreased internode elongation, plant tillering, and/or stomatal density changes that are observed in plants subjected to suppression of MSH1, but these phrases are applicable to plants with these phenotypes regardless of how the plants were produced.

As used herein, the phrases “mutated chromosomal loci” (plural) (plural), “mutated chromosomal locus” (singular), “chromosomal mutations” and “chromosomal mutation” refer to portions of a chromosome that have undergone a heritable genetic change in a nucleotide sequence relative to the nucleotide sequence in the corresponding parental chromosomal loci. Mutated chromosomal loci comprise mutations that include, but are not limited to, nucleotide sequence inversions, insertions, deletions, substitutions, or combinations thereof. In certain embodiments, the mutated chromosomal loci can comprise mutations that are reversible. In this context, reversible mutations in the chromosome can include, but are not limited to, insertions of transposable elements, defective transposable elements, and certain inversions. In certain embodiments, the chromosomal loci comprise mutations are irreversible. In this context, irreversible mutations in the chromosome can include, but are not limited to, deletions.

As used herein, the phrases “mutated gene” or “gene mutation” or “mutant” or “mutant thereof” refer to portions of a gene that have undergone a heritable genetic change in a nucleotide sequence relative to the nucleotide sequence in the corresponding parental gene that results in a reduction in function of the gene's encoded protein function. Mutations include, but are not limited to, nucleotide sequence inversions, insertions, deletions, substitutions, or combinations thereof. In certain embodiments, the mutated gene can comprise mutations that are reversible. In this context, reversible mutations in the chromosome can include, but are not limited to, insertions of transposable elements, defective transposable elements, and certain inversions. In certain embodiments, the gene comprises mutations are irreversible. In this context, irreversible mutations in the chromosome can include, but are not limited to, deletions.

As used herein, the phrase “new combinations of altered chromosomal loci” refers to nuclear chromosomal regions in a progeny plant with one or more differences in altered chromosomal loci when compared to altered chromosomal loci of a parental plant if derived by self-pollination, or if derived from a cross, when compared to either parental plant, each compared separately to said progeny plant.

As used herein, the term “non-regenerable” refers to a plant part or plant cell that can not give rise to a whole plant.

As used herein, the phrase “obtaining a nucleic acid associated with the altered chromosomal locus” refers to any method that provides for the physical separation or enrichment of the nucleic acid associated with the altered chromosomal locus from covalently linked nucleic acid that have not been altered. In this context, the nucleic acid does not necessarily comprise the alteration (i.e. such as methylation) but at least comprises one or more of the nucleotide base or bases that are altered. Nucleic acids associated with an altered chromosomal locus can thus be obtained by methods including, but not limited to, molecular cloning, PCR, or direct synthesis based on sequence data. Once identified, the sequence information that identifies a nucleic acid associated with the altered chromosomal locus can be used in methods that measure the altered chromosome as this sequence.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the terms “pericentromeric” or “pericentromere” refer to heterochromatic regions containing abundant repeated sequences, transposable elements, and retrotransposons that physically flank the centromeric regions. At the sequence level, a functional definition for pericentromeric sequences are highly repeated sequences that contain transposable elements and retrotransposons embedded in said repeated sequences. When known, centromeric repeats can be computationally removed from the repeated sequences, but their presence is not detrimental if not computationally removed. When available, chromosomal positioning information about the location of sequences that are located adjacent to the centromere can be used as an additional criteria for pericentromeric sequences.

As used herein, the terms “polynucleotide,” “nucleic acid”, “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; inter-nucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970):

As used herein, the term “progeny” refers to any one of a first, second, third, or subsequent generation obtained from a parent plant if self pollinated or from parent plants if obtained from a cross, or through any combination of selfing and crossing. Any materials of the plant, including but not limited to seeds, tissues, pollen, and cells can be used as sources of RNA or DNA for determining the status of the RNA or DNA composition of said progeny.

As used herein, the phrase “reference plant” refers to a parental plant or progenitor of a parental plant prior to expression of a recombinant DNA methyltransferase, but otherwise isogenic to the candidate or test plant to which it is being compared. In a cross of two parental plants, a “reference plant” can also be from parental plants wherein expression of a recombinant DNA methyltransferase was not used in said parental plants or their progenitors.

As used herein, the phrase “quantitative variation” or “V_{Q}” refers to phenotypic variation that is observed in individual progeny lines derived from outcrosses of plants where expression of a recombinant DNA methyltransferase occurred and that exhibit discrete variation to other plants.

As used herein, the term “S1” refers to a first selfed plant. “S2” refers to progeny from the self pollination of the S1 plant. “S3” refers to progeny from the self pollination of the S2 plant. “S4” refers to progeny from the self pollination of the S3 plant. “S5” refers to progeny from the self pollination of the S4 plant. “Sn” refers to progeny from the self pollination of the S(n−1) plant, where “n” is the number of generations starting from the initial S1 cross.

As used herein, the terms “self”, “selfing”, or “selfed” refer to the process of self pollinating a plant.

As used herein the phrase “recombinant DNA methyltransferase” refers to any protein or gene encoding a protein that has DNA methyltransferase activity capable of methylating cytosine residues in DNA (C bases in DNA) at CHG and/or CHH sequences, and may or may not also methylate DNA at CG positions (Arabidopsis Met! and its orthologs are CG DNA methyltransferases and therefore are not included in the term “recombinant DNA methyltransferase” herein). Recombinant DNA methyltransferases include, but are not limited to, the DRM2 group and CMT3 group of DNA methyltransferases and proteins or fusion proteins that contain catalytic domains of the DRM2 group and CMT3 group of DNA methyltransferases. In certain embodiments a DNA binding protein, including RNA-guided binding proteins such as CRISPR/CAS9 that bind DNA or KYP proteins that bind DNA, are fused to at either the N-terminus or C-terminus, with or without flexible peptide linkers such as GGGSS or GGSS or other flexible linkers used in protein fusions, of the catalytic domains of the DRM2 group and CMT3 group of DNA methyltransferases. Recombinant DNA methyltransferase include, but are not limited to, DNA sequence changes to endogenous DNA methyltransferases at their native positions in the plant's chromosome if their expression levels are increased through these sequence changes or if a non-native coding region is inserted into the endogenous coding region or if promoter elements are inserted into their promoter regions or enhancers are inserted upstream, downstream, or withing introns, of the endogenous DNA methyltransferase.

As used herein, the phrases “suppression” or “suppressing expression” of a gene refer to any genetic, nucleic acid, nucleic acid analog, environmental manipulation, grafting, transient or stably transformed methods of any of the aforementioned methods, or chemical treatment that provides for decreased levels of functional gene activity in a plant or plant cell relative to the levels of functional gene activity that occur in an otherwise isogenic plant or plant cell that had not been subjected to this genetic or environmental manipulation.

As used herein, the term “transgene” or “transgenic” refers to any recombinant DNA that has been transiently introduced into a cell or stably integrated into a chromosome or minichromosome that is stably or semi-stably maintained in a host cell. In this context, sources for the recombinant DNA in the transgene include, but are not limited to, DNAs from an organism distinct from the host cell organism, species distinct from the host cell species, varieties of the same species that are either distinct varieties or identical varieties, DNA that has been subjected to any in vitro modification, in vitro synthesis, recombinant DNA, and any combination thereof. The terms transgene or transgenic include inserting or changing DNA sequences at endogenous genes to alter their expression or function through any non-natural process.

As used herein, the phrases “useful for plant breeding” or “useful for breeding” refer to plants derived from one or more progenitor plants or plant cells that were subjected to expression of a recombinant DNA methyltransferase that are useful in a plant breeding program for the objecting of developing improved plants and plant seeds to a greater extent than control plants not subjected to expression of a recombinant DNA methyltransferase or derived from progenitor plants subjected to expression of a recombinant DNA methyltransferase.

As used herein, the phrases “useful trait” or “useful traits” refer to plants derived from one or more progenitor plants that were subjected to expression of a recombinant DNA methyltransferase that exhibit one or more agriculturally useful traits to a greater extent than control plants not subjected to expression of a recombinant DNA methyltransferase or derived from progenitor plants subjected to expression of a recombinant DNA methyltransferase.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Methods for introducing heritable epigenetic variation that result in plants that exhibit useful traits are provided herewith along with plants, plant seeds, plant parts, plant cells, and processed plant products obtainable by these methods. In certain embodiments, methods provided herewith can be used to introduce epigenetic variation into varietal or non-hybrid plants that result in useful traits as well as useful plants, plant parts including, but not limited to, seeds, plant cells, and processed plant products that exhibit, carry, or otherwise reflect benefits conferred by the useful traits. In other embodiments, methods provided herewith can be used to introduce epigenetic variation into plants that are also amenable to hybridization. Related methods are available in U.S. Patent Application No. 20120284814, U.S. Provisional Application 61/863,267, U.S. Provisional Application 61/882,140, and U.S. Provisional Application 61/901,349, U.S. Provisional Application 61/930602, U.S. Provisional Application 61/970424, U.S. Provisional Application 61/980096, and U.S. Provisional 61/983,520, each of which is incorporated by reference in its entirety, except that the claims and definitions sections are excluded from incorporation).

In certain embodiments, methods for selectively expressing recombinant DNA methyltransferases in sub-populations of cells found in plants that contain plastids referred to herein as “sensory plastids” are provided. Sensory plastids are plastids that occur in cells that exhibit preferential expression of at least the Msh1 promoter. In certain embodiments, Msh1 and other promoters active in sensory plastids can thus be operably linked to a heterologous sequence to express a recombinant DNA methyltransferase in cells containing the sensory plastids. In addition to the distinguishing characteristic of expressing MSH1, such cells containing sensory plastids can also be readily identified as their plastids are only about 30-40% of the size of the chloroplasts contained within mesophyll cells. Other promoters active in sensory plastids include, but are not limited to, PPD3 and PSBO1 gene promoters. Selective functional expression of a recombinant DNA methyltransferase in cells containing sensory plastids can trigger epigenetic changes that provide useful plant traits.

Identification of DRM2 Group or CMT3 Group DNA Methyltransferases

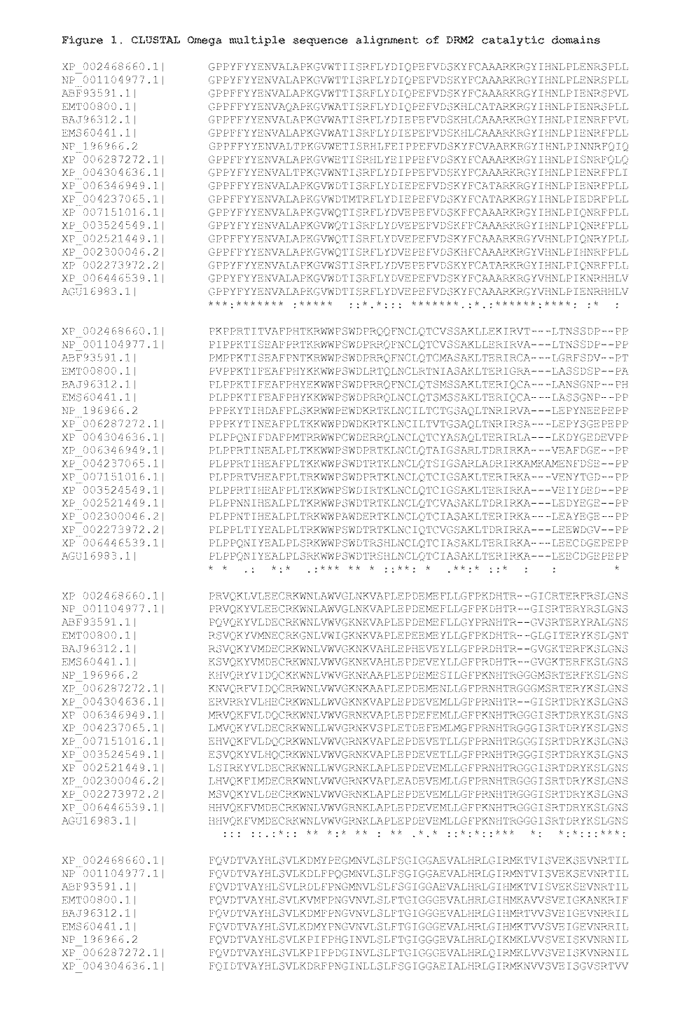

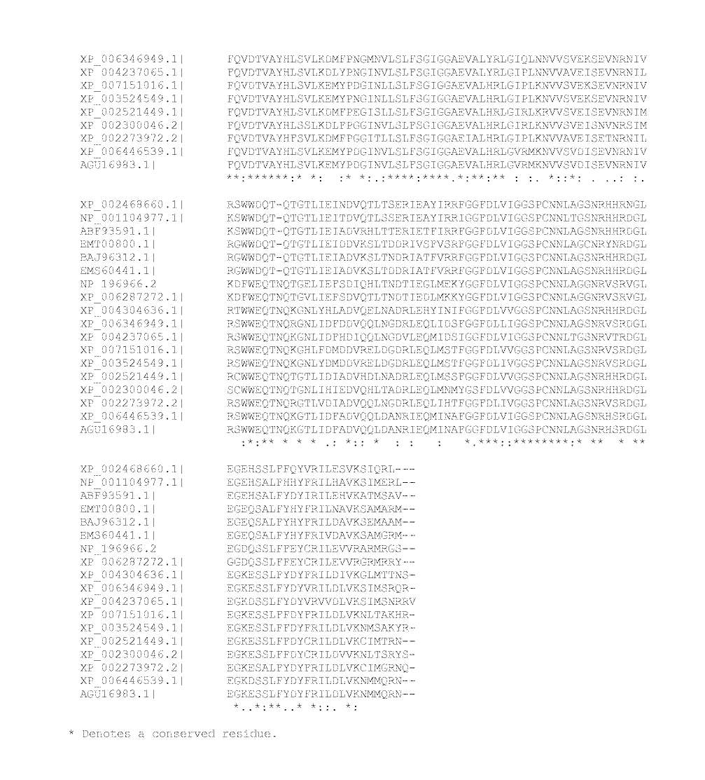

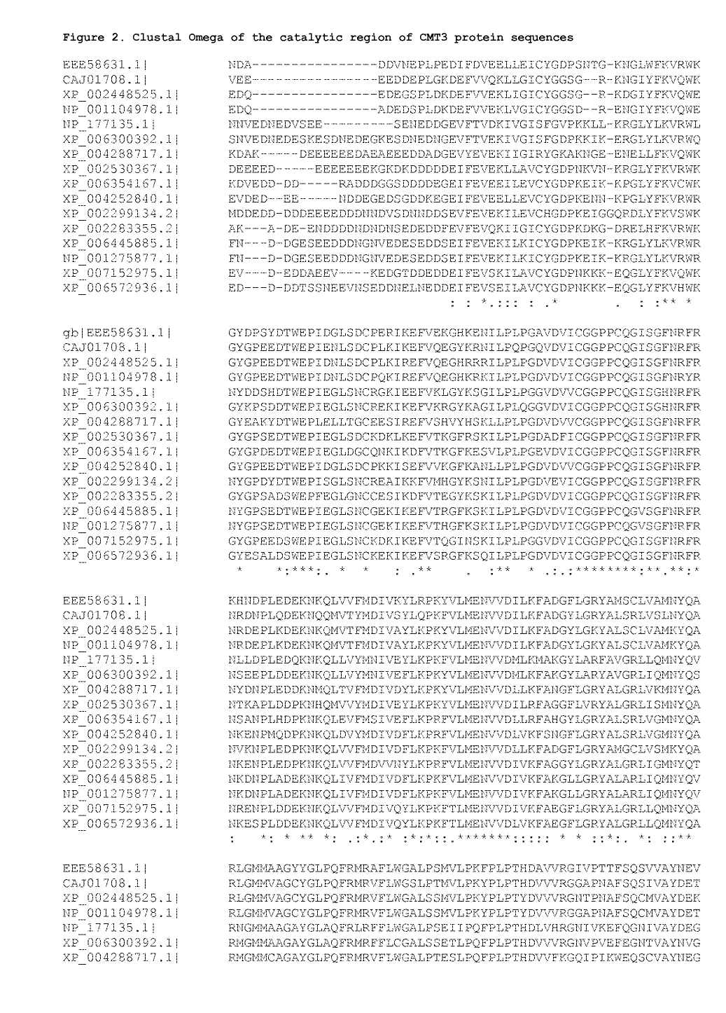

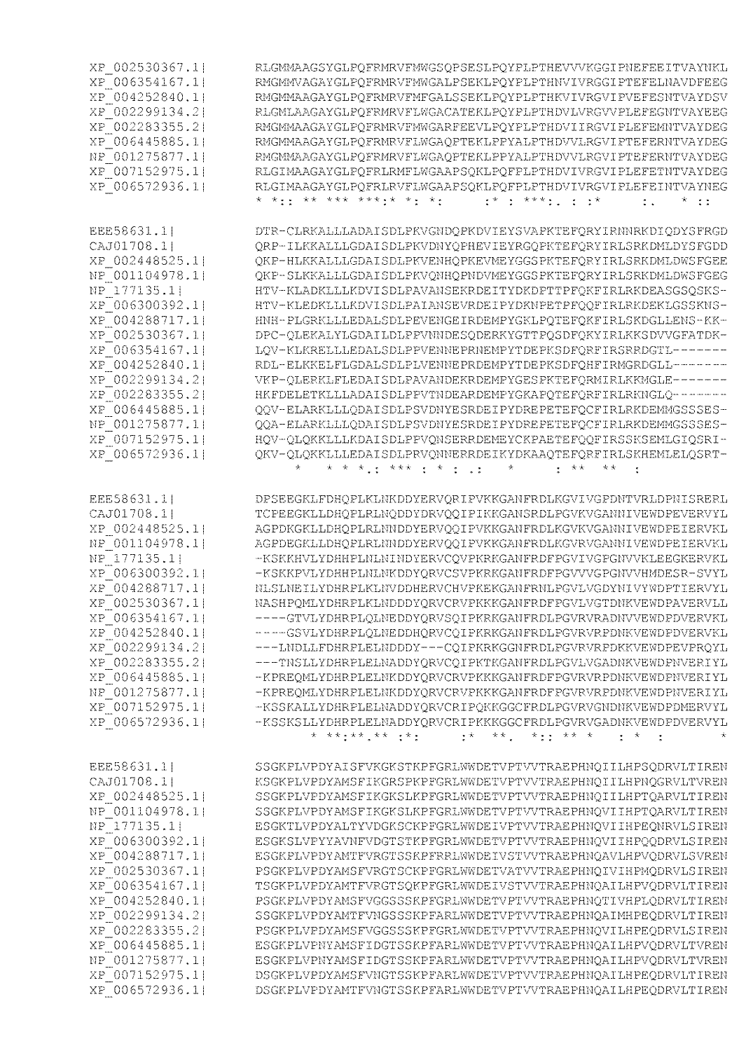

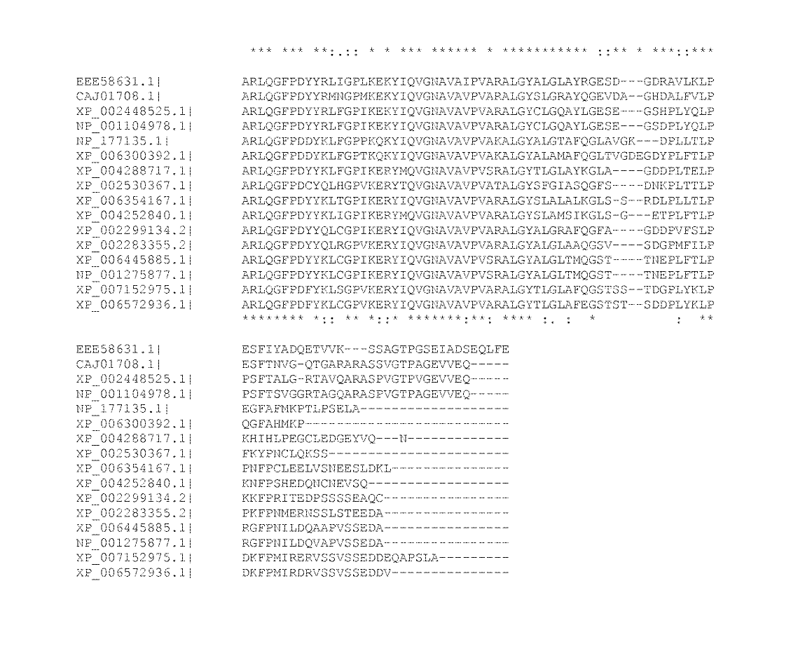

Orthologous DRM2 or CMT3 or other DNA methyltransferase genes related to DRM2 or CMT3 genes can be obtained from many crop species through the BLAST comparison of the protein sequences of the DRM2 or CMT3 genes in Table 1 to the genomic databases (NCBI and publically available genomic databases for specific crop species), as well as from the specific names of the subunits. Specifically the genome, cDNA, or EST sequences are available for apples, beans, barley, Brassica napus, rice, Cassava, Coffee, Eggplant, Orange, sorghum, tomato, cotton, grape, lettuce, tobacco, papaya, pine, rye, soybean, sunflower, peach, poplar, scarlet bean, spruce, cocoa, cowpea, maize, onion, pepper, potato, radish, sugarcane, wheat, and other species at the following internet or world wide web addresses: “compbio.dfci.harvard.edu/tgi/plant.html”; “genomevolution.org/wiki/index.php/Sequenced_plant_genomes”; “ncbi.nlm.nih.gov/genomes/PLANTS/PlantList.html”; “plantgdb.org/”; “arabidopsis.org/portals/genAnnotation/other_genomes/”;“gramene.org/resources/”; “genomenewsnetwork.org/resources/sequenced_genomes/genome_guide_p1.shtml”; “jgi.doe.gov/programs/plants/index.jsf”; “chibba.agtec.uga.edu/duplication/”; “mips.helmholtz-muenchen.de/plant/genomes.jsp”; “science.co.il/biomedical/Plant-Genome-Databases.asp”; “jcvi.org/cms/index.php?id=16”; and “phyto5.phytozome.net/Phytozome_resources.php”. Candidate genes or proteins can be aligned by BLAST or Clustal Omega to identify identical amino acids at the positions indicated in FIGS. 1 and 2. Candidate genes or proteins with 25%-50%, 50%-70%, 70%-80%, 80%-90%, 90%-95%, or 95%-100% identity at these positions and that have DNA methyltransferase activity are considered DRM2 group or CMT3 group DNA methyltransferases. Conservatively modified variants of these DRM2 group or CMT3 group DNA methyltransferases occur naturely or can be intentially modified by recombinant DNA methods and still be contemplated by the present invention.

Methods for obtaining DNA methyltransferase genes for include, but are not limited to, techniques such as: i) searching amino acid and/or nucleotide sequence databases comprising sequences from the plant species to identify the DNA methyltransferases genes by sequence identity comparisons; ii) cloning the DNA methyltransferases gene by either PCR from genomic sequences or RT-PCR from expressed RNA; iii) cloning the DNA methyltransferases target gene from a genomic or cDNA library using PCR and/or hybridization based techniques; iv) cloning the DNA methyltransferases target gene from an expression library where an antibody directed to the DNA methyltransferases target gene protein is used to identify the DNA methyltransferases target gene containing clone; v) cloning the DNA methyltransferases target gene by complementation of an DNA methyltransferases target gene mutant or DNA methyltransferases gene deficient plant; or vi) any combination of (i), (ii), (iii), (iv), and/or (v). The DNA sequences of the target genes can be obtained from the promoter regions or transcribed regions of the target genes by PCR isolation from genomic DNA, or PCR of the cDNA for the transcribed regions, or by commercial synthesis of the DNA sequence. RNA sequences can be chemically synthesized or, more preferably, by transcription of suitable DNA templates. Confirming that the candidate DNA methyltransferases target gene can methylate DNA can be readily determined or confirmed by constructing a plant transformation vector that provides for expression of the target gene, transforming the plants with the vector, and determining if plants transformed with the vector exhibit increased DNA methylation. Additionally, diagnostic phenotypes include those that are typically observed in various plant species when epigenetic marks are perturbed, including leaf variegation, cytoplasmic male sterility (CMS), a reduced growth-rate phenotype, delayed or non-flowering phenotype, and enhanced susceptibility to pathogens. These characteristic responses have been described previously as developmental reprogramming or “MSH1-dr” (Xu et al. Plant Physiol. Vol. 159:711-720, 2012).

In certain embodiments, the recombinant polynucleotides of the invention encode DNA methyltransferase polypeptides having at least about 25%-50%, 50%-70%, 70%-80%, 80%-90%, 90%-95%, or 95%-100% amino acid residue sequence identity to the amino acids that are identified in FIG. 1 or 2 as being identical (*) in the catalytic domains of the multiple catalytic domains analyzed in those Figures. In preferred embodiments, the polynucleotides of the invention encode polypeptides having at least about 80%-90%, 90%-95%, or 95%-100% amino acid residue sequence identity to the amino acids that are identified in FIG. 1 or 2 as being identical (*) in the catalytic domains of the multiple catalytic domains analyzed in those Figures. In certain embodiments polynucleotides of the invention further include polynucleotides that encode conservatively modified variants of polypeptides encoded by the genes or proteins listed in Tables 1, and homologous or orthologous genes or proteins of other plant species, more preferably of any crop species. In certain embodiments, the recombinant polynucleotides of the invention encode conservatively modified variants amino acids of DNA methyltransferase polypeptides at the amino acids positions that are identified in FIG. 1 or 2 as being identical (*) in the catalytic domains of the multiple catalytic domains analyzed in those Figures, and if the conservatively modified variants are considered to be equivalent to an identical (*) amino acid, then in certain embodiments, the recombinant polynucleotides of the invention encode DNA methyltransferase polypeptides having at least about 25%-50%, 50%-70%, 70%-80%, 80%-90%, 90%-95%, or 95%-100% amino acid residue sequence identity to the amino acids that are identified in FIG. 1 or 2 as being identical (*) in the catalytic domains of the multiple catalytic domains analyzed in those Figures. In certain embodiments genes encoding mutant DNA methyltransferase proteins have insertions or deletions of one or more amino acids while having at least about 25%-50%, 50%-70%, 70%-80%, 80%-90%, 90%-95%, or 95%-100% amino acid residue sequence identity to the amino acids that are identified in FIG. 1 or 2 as being identical (*) in the catalytic domains of the multiple catalytic domains analyzed in those Figures.

In general, methods provided herewith for introducing epigenetic variation in plants require plants or plant cells to be subjected to expression of a recombinant DNA methyltransferase for a time sufficient in leaves or in appropriate subsets of cells (i.e cells containing sensory plastids). As such, a wide variety of methods of expressing a recombinant DNA methyltransferase can be employed to practice the methods provided herewith and the methods are not limited to a particular expression technique.

In certain embodiments, recombinant DNA methyltransferase genes may be used directly in either a homologous or a heterologous plant species to provide for expression of a recombinant DNA methyltransferase gene in either the homologous or heterologous plant species. An transgene from Arabidopsis or rice or other plant species listed in Table 1 that provides for a expression of a recombinant DNA methyltransferase can be used in certain embodiments in millet, sorghum, and maize, or other plants including, but not limited to, cotton, canola, wheat, barley, flax, oat, rye, turf grass, sugarcane, alfalfa, banana, broccoli, cabbage, carrot, cassava, cauliflower, . celery, citrus, a cucurbit, eucalyptus, garlic, grape, onion, lettuce, pea, peanut, pepper, potato, poplar, pine, sunflower, safflower, soybean, strawberry, sugar beet, sweet potato, tobacco, cassava, cauliflower, celery, citrus, cotton, a cucurbit, eucalyptus, garlic, grape, onion, lettuce, pea, peanut, pepper, potato, poplar, pine, sunflower, safflower, strawberry, sugar beet, sweet potato, tobacco, cassava, cauliflower, celery, citrus, cucurbits, eucalyptus, garlic, grape, onion, lettuce, pea, peanut, pepper, poplar, pine, sunflower, safflower, soybean, strawberry, sugar beet, tobacco, Jatropha, Camelina, and Agave.

Inducible recombinant DNA methyltransferase expression can be with promoters that include, but are not limited to, a PR-1a promoter (US Patent Application Publication Number 20020062502) or a GST II promoter (WO 1990/008826 A1). Additional examples of inducible promoters include, without limitation, the AdhI promoter which is inducible by hypoxia or cold stress, the Hsp70 promoter which is inducible by heat stress, and the PPDK promoter which is inducible by light. In other embodiments, a transcription factor that can be induced or repressed as well as a promoter recognized by that transcription factor and operably linked to the recombinant DNA methyltransferase sequences are provided. Such transcription factor/promoter systems include, but are not limited to: i) RF2a acidic domain-ecdysone receptor transcription factors/cognate promoters that can be induced by methoxyfenozide, tebufenozide, and other compounds (US Patent Application Publication Number 20070298499); ii) chimeric tetracycline repressor transcription factors/cognate chimeric promoters that can be repressed or de-repressed with tetracycline (Gatz, C., et al. (1992). Plant J. 2, 397-404), estradiol or dexamethasone inducible promoters (Aoyama and Chua The Plant Journal (1997) 11(3):605-612; Zuo et al., The Plant Journal (2000) 24(2):265-273), and the like.

In certain embodiments, a promoter that provides for selective expression of a recombinant DNA methyltransferase in cells containing sensory plastids is used. In certain embodiments, this promoter is an Msh1 or a PPD3 promoter. In certain embodiments, this promoter is an Msh1 or PPD3 or PSBO1 or PSBO2 promoter and an operably linked recombinant DNA methyltransferase gene, or fragment thereof, that is provided in Table 1. Msh1 promoters that can be used to express recombinant DNA methyltransferase in cells containing sensor plastids include, but are not limited to, Arabidopsis, sorghum, tomato, rice, and maize Msh1 promoters as well as functional derivatives thereof that likewise provide for expression in cells that contain sensor plastids. In certain embodiments, 5′ deletion derivatives of the Msh1 promoters comprising promoter sizes of about 1500 Bp, 1000 Bp, or about 750 Bp of can also be used to express recombinant DNA methyltransferases. In certain embodiments, recombinant DNA constructs for expression of recombinant DNA methyltransferase can comprise a MSH1 or PPD3 or PSBO1 or PSBO2 promoter from a dicotyledonous species such as Arabidopsis, soybeans or canola, or monocotyledonous species such as rice, maise or sorghum operably attached to a recombinant DNA methyltransferase followed by a polyadenylation region. Various 3′ polyadenylation regions known to function in monocots and dicot plants include, but are not limited to, the Nopaline Synthase (NOS) 3′ region, the Octapine Synthase (OCS) 3′ region, the Cauliflower Mosaic Virus 35S 3′ region, the Mannopine Synthase (MAS) 3′ region. In certain embodiments recombinant DNA constructs for expression of monocot target genes can comprise MSH1 or PPD3 promoter from a monocot species such as rice, maize, sorghum or wheat can either be attached to a monocot intron before the recombinant DNA methyltransferase coding region. Monocot introns that are beneficial to gene expression when located between the promoter and coding region are the first intron of the maize ubiquitin (described in U.S. Pat. No. 6,054,574) and the first intron of rice actin 1 (McElroy, Zhang et al. Plant Cell 2(2): 163-171, 1990). Additional introns that are beneficial to gene expression when located between the promoter and coding region are the maize hsp70 intron (described in U.S. Pat. No. 5,859,347), and the maize alcohol dehydrogenase 1 genes introns 2 and 6 (described in U.S. Pat. No. 6,342,660).

In still other embodiments, transgenic plants are provided wherein the transgene that provides for recombinant DNA methyltransferase expression is flanked by sequences that provide for removal for the transgene. Such sequences include, but are not limited to, transposable element or recombinase sequences that are acted on by a cognate transposase or recombinase. Non-limiting examples of such recombinase systems that have been used in transgenic plants include the cre-lox and FLP-FRT systems.

Recombinant DNA methyltransferase gene expression can be readily identified or monitored by molecular techniques. Molecular methods for monitoring recombinant DNA methyltransferase target gene RNA expression levels include, but are not limited to, use of semi-quantitive or quantitative reverse transcriptase polymerase chain reaction (qRT-PCR) techniques. The use of semi-quantitive PCR techniques to monitor target gene suppression has been described (Sandhu et al. 2007). Various quantitative RT-PCR procedures including, but not limited to, TaqMan™ reactions (Applied Biosystems, Foster City, Calif. US), use of Scorpion™ or Molecular Beacon™ probes, or any of the methods disclosed in Bustin, S. A. (Journal of Molecular Endocrinology (2002) 29, 23-39) can be used. It is also possible to use other RNA quantitation techniques such as Quantitative Nucleic Acid Sequence Based Amplification (Q-NASBA™) or the Invader™ technology (Third Wave Technologies, Madison, Wis.).

Mutations or alterations of endogenous plant DNA methyltransferase target genes to produce recombinant DNA methyltransferase genes can be obtained from a variety of sources and by a variety of techniques. A homologous replacement sequence containing one or more alterations and homologous sequences at both ends of the double stranded break can provide for homologous recombination and substitution of the resident wild-type DNA methyltransferase target gene sequence in the chromosome with a replacement sequence with the gain of function mutation(s). Such gain of function mutations include, but are not limited to, insertions, deletions, and substitutions of sequences within DNA methyltransferase target gene that result in sufficient gain of function to elicit alterations (i.e. heritable and reversible epigenetic changes) in other chromosomal loci or mutations in other chromosomal loci. Gain of function alterations include, but are not limited to, overexpression of the target gene or fragments thereof and/or fusions of DNA binding proteins, including CRISPR-CAS9 types, to the endogenous DNA methyltransferase.

Methods for substituting endogenous chromosomal sequences by homologous double stranded break repair have been reported in tobacco and maize (Wright et al., Plant J. 44, 693, 2005; D'Halluin, et al., Plant Biotech. J. 6:93, 2008). A homologous replacement can also be introduced into a targeted nuclease cleavage site by non-homologous end joining or a combination of non-homologous end joining and homologous recombination (reviewed in Puchta, J. Exp. Bot. 56, 1, 2005; Wright et al., Plant J. 44, 693, 2005). In certain embodiments, at least one site specific double stranded break can be introduced into the endogenous DNA methyltransferase gene by a meganuclease. Genetic modification of meganucleases can provide for meganucleases that cut within a recognition sequence that exactly matches or is closely related to specific endogenous DNA methyltransferase gene sequence (WO/06097853A1, WO/06097784A1, WO/04067736A2, U.S. 20070117128A1). It is thus anticipated that one can select or design a nuclease that will cut within a target DNA methyltransferase target gene sequence. In other embodiments, at least one site specific double stranded break can be introduced in the endogenous DNA methyltransferase target gene target sequence with a zinc finger nuclease. The use of engineered zinc finger nuclease to provide homologous recombination in plants has also been disclosed (WO 03/080809, WO 05/014791, WO 07014275, WO 08/021207). In still other embodiments, CRISPR/CAS9 systems are used for genome editing to create mutations or gene replacement and modifications alterations (Strauβ and Lahaye, Mol Plant. 2013 September: 6(5):1384-7; Sampson and Weiss Bioessays 2014 January; 36(1):34-8). In still other embodiments, alterations in endogenous DNA methyltransferase target genes can be identified through use of the TILLING technology (Targeting Induced Local Lesions in Genomes) as described by Henikoff et al. where traditional chemical mutagenesis would be followed by high-throughput screening to identify plants useful point mutations or other mutations in the endogenous DNA methyltransferase target gene (Henikoff et al., Plant Physiol. 2004, 135:630-636).

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., Nature. 2005, 10; 433(7026):629-33. It is further understood that the recombinant DNA constructs can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the recombinant DNA constructs into the chromosome of a plant cell, a plant tissue or a plant.

Methods of introducing plant minichromosomes comprising plant centromeres that provide for the maintenance of the recombinant minichromosome in a transgenic plant can also be used in practicing this invention (U.S. Pat. No. 6,972,197 and US Patent Application Publication 20120047609). In these embodiments of the invention, the transgenic plants harbor the minichromosomes as extrachromosomal elements that are not integrated into the chromosomes of the host plant. It is anticipated that such mini-chromosomes may be useful in providing for variable transmission of a resident recombinant DNA construct that expresses a recombinant DNA methyltransferase.

Methods where recombinant DNA methyltransferase expression or genome edited expression or alteration is effected in cultured plant cells are also provided herein. In certain embodiments, recombinant DNA methyltransferase expression or genome edited expression or alteration is effected in cultured plant cells by introducing a nucleic acid that provides for such expression in the plant cells. Nucleic acids that can be used to provide for expression in cultured plant cells include, but are not limited to, transgenes, mRNA, and recombinant virus vectors.

Nucleic acid or protein molecules that provide DNA methyltransferase activity can be introduced by electroporation or particle gun or other physical methods or Agrobacterium or Rhizobium gene transfer methods. The expression of the plant recombinant DNA methyltransferase genes listed in Table 1 in cultured plant cells is specifically provided herein.

Recombinant DNA methyltransferase expression can also be readily identified or monitored by traditional methods where plant phenotypes are observed. For example, recombinant DNA methyltransferase gene function can be identified or monitored by observing epigenetic effects that include leaf variegation, cytoplasmic male sterility (CMS), a reduced growth-rate phenotype, delayed or non-flowering phenotype, and/or enhanced susceptibility to pathogens. Phenotypes indicative of epigenetic phenotypes in various plants are provided in WO 2012/151254, which is incorporated herein by reference in its entirety. These phenotypes that are associated with epigenetic phenotypes are referred to herein as “discrete variation” (V_{D}). Epigenetic variation can also produce changes in plant tillering, height, internode elongation and stomatal density (referred to herein as “MSH1-dr” phenotypes) that can be used to identify or monitor epigenetic effects in plants. Other biochemical and molecular traits can also be used to identify or monitor epigenetic effects in plants. Such molecular traits can include, but are not limited to, changes in expression of genes involved in cell cycle regulation, Giberrellic acid catabolism, auxin biosynthesis, auxin receptor expression, flower and vernalization regulators (i.e. increased FLC and decreased SOC1 expression), as well as increased miR156 and decreased miR172 levels. Such biochemical traits can include, but are not limited to, up-regulation of most compounds of the TCA, NAD and carbohydrate metabolic pathways, down-regulation of amino acid biosynthesis, depletion of sucrose in certain plants, increases in sugars or sugar alcohols in certain plants, as well as increases in ascorbate, alphatocopherols, and stress-responsive flavones apigenin, and apigenin-7-oglucoside, isovitexin, kaempferol 3-O-beta-glucoside, luteolin-7-O-glucoside, and vitexin. It is further contemplated that in certain embodiments, a combination of both molecular, biochemical, and traditional methods can be used to identify or monitor epigenetic effects in plants. It is further contemplated that in certain embodiments, plants displaying one or more Msh1-dr phenotypes in at least a portion of said plants can be outcrossed or selfed to obtain progeny plants lacking recombinant DNA methyltransferase genes or proteins and exhibiting enhanced growth or yields or useful traits in the F1, F2, F3, or Fn generations.

Expression of recombinant DNA methyltransferase that results in useful epigenetic changes and useful traits can also be readily identified or monitored by assaying for characteristic DNA methylation and/or gene transcription and/or sRNA patterns that occur in plants subject to such perturbations. In certain embodiments, characteristic DNA methylation and/or gene transcription and/or sRNA patterns that occur in plants subject to expression of recombinant DNA methyltransferase can be monitored in a plant, a plant cell, plants, seeds, and/or processed products obtained therefrom to identify or monitor effects mediated by expression of a recombinant DNA methyltransferase. Expression of recombinant DNA methyltransferase results in: hypermethylation of CG, CHG, and CHH chromosomal positions and regions. In certain embodiments, expression of recombinant DNA methyltransferase in the plant species being analyzed for DNA methylation changes provides altered chromosomal loci with altered DNA methylation patterns. In certain embodiments, first or second or later generation progeny of a plant subjected to expression of a recombinant DNA methyltransferase will exhibit CG differentially methylated regions (DMR) of various discrete chromosomal loci that include, but are not limited to, the MSH1 locus and changes in plant defense and stress response gene expression. In certain embodiments, a plant, a plant cell, a seed, plant populations, seed populations, and/or processed products obtained therefrom that has been subject to expression of a recombinant DNA methyltransferase will exhibit pericentromeric or repeated sequence or transposable element CHG and/or CHH hypermethylation and/or CG hypermethlation of various discrete or localized chromosomal regions. Such CHG and/or CHH hypermethylation is understood to be methylation at the sequence “CHG” or “CHH” where H=A, T, or C. Such CG and CHG and CHH hypermethylation can be assessed by comparing the methylation status of a sample from plants or seed that had been subjected to expression of a recombinant DNA methyltransferase, or a sample from progeny plants or seed derived therefrom, to a sample from control plants or seed that had not been subjected to expression of a recombinant DNA methyltransferase. It is further contemplated that in certain embodiments, plants subjected to expression of a recombinant DNA methyltransferase displaying altered chromosomal loci in at least a portion of said plants can be outcrossed or selfed to obtain progeny plants lacking a recombinant DNA methytransferase and exhibiting enhanced growth or yields or useful traits in the F1, F2, F3, or Fn generations.

A variety of methods that provide for functional expression of a recombinant DNA methyltransferase in a plant followed by recovery of progeny plants not expressing a recombinant DNA methyltransferase and with useful epigenetic changes are provided herein. In certain embodiments, progeny plants can be recovered by downregulating expression of a recombinant DNA methyltransferase or by removing the recombinant DNA methyltransferase transgene with a transposase or recombinase. In certain embodiments of the methods provided herein, a recombinant DNA methyltransferase gene is functionally suppressed or removed from a target plant or plant cell and progeny plants by genetic techniques. In one exemplary and non-limiting embodiment, progeny plants can be obtained by selfing a plant that is heterozygous for the transgene that provides for expression of a recombinant DNA methyltransferase by segregation. Selfing of such heterozygous plants (or selfing of heterozygous plants regenerated from plant cells) provides for the transgene to segregate out of a subset of the progeny plant population. Where a recombinant DNA methyltransferase gene is derived by a dominant mutation in an endogenous gene the plant can, in yet another exemplary and non-limiting embodiment, be selfed if heterozygous or crossed to wild-type plants if homozygous and then selfed to obtain progeny plants that are homozygous for a functional, wild-type DNA methyltransferase gene allele. In other embodiments, plant cell and/or progeny plants that lack expression of or lack the recombinant DNA methyltransferase gene are recovered by molecular genetic techniques. Non limiting and exemplary embodiments of such molecular genetic techniques include: i) dowriregulation of expression under the control of a regulated promoter by withdrawal of an inducer required for activity of that promoter or introduction and/or induction of a repressor of that promoter; or, ii) exposure of the transgene flanked by transposase or recombinase recognition sites to the cognate transposase or recombinase that provides for removal of that transgene.

In certain embodiments of the methods provided herein, progeny plants derived from plants subjected to functional expression of a recombinant DNA methyltransferase exhibit male sterility, dwarfing, variegation, and/or delayed flowering time and lack a recombinant DNA methyltransferase gene are obtained and maintained as independent breeding lines or as populations of plants. It has been found that such phenotypes appear to sort, so that it is feasible to select a cytoplasmic male sterile plant displaying normal growth rate and no variegation, for example, or a stunted, male fertile plant that is highly variegated. We refer to this phenomenon herein as discrete variation (VD). It is further contemplated that such individual lines that exhibit discrete variation (VD) can be obtained by any of the aforementioned genetic techniques, molecular genetic techniques, or combinations thereof.

Individual lines obtained from plants subjected to expression of a recombinant DNA methyltransferase that exhibit discrete variation (V_{D}) can be crossed to other plants to obtain progeny plants that lack the phenotypes associated with discrete variation (V_{D}) (i.e. male sterility, dwarfing, variegation, and/or delayed flowering time). In certain embodiments, progeny of such outcrosses can be selfed to obtain individual progeny lines that exhibit significant useful phenotypic variation and/or useful traits. Such phenotypic variation that is observed in these individual progeny lines derived from outcrosses of plants subjected expression of a recombinant DNA methyltransferase and that exhibit discrete variation to other plants is herein referred to as “quantitative variation” (V_{Q}). Certain individual progeny plant lines obtained from the outcrosses of plants where expression of a recombinant DNA methyltransferase occurred to other plants can exhibit useful phenotypic variation where one or more traits are improved relative to either parental line and can be selected. Useful phenotypic variation that can be selected in such individual progeny lines includes, but is not limited to, increases in fresh and dry weight biomass and/or seed or fruit yield relative to either parental line.

Individual lines obtained from plants wherein expression of a recombinant DNA methyltransferase occurred that exhibit discrete variation (V_{D}) can also be selfed to obtain progeny plants that lack the phenotypes associated with discrete variation (V_{D}) (i.e. male sterility, dwarfing, variegation, and/or delayed flowering time). Recovery of such progeny plants that lack the undesirable phenotypes can in certain embodiments be facilitated by removal of the transgene or endogenous locus that provides for expression of a recombinant DNA methyltransferase. In certain embodiments, progeny of such selfs can be used to obtain individual progeny lines or populations that exhibit significant useful phenotypic variation. Certain individual progeny plant lines or populations obtained from selfing plants where expression of a recombinant DNA methyltransferase occurred can exhibit useful phenotypic variation where one or more traits are improved relative to the parental line that was not subjected to expression of a recombinant DNA methyltransferase can be selected. Useful phenotypic variation that can be selected in such individual progeny lines includes, but is not limited to, increases in fresh and dry weight biomass and/or yield relative to the parental line.

In certain embodiments, an outcross of an individual line exhibiting discrete variability can be to a plant that has not been subjected to expression of a recombinant DNA methyltransferase but is otherwise isogenic to the individual line exhibiting discrete variation. In certain exemplary embodiments, a line exhibiting discrete variation is obtained by expression of a recombinant DNA methyltransferase in a given germplasm and outcrossing to a plant having that same germplasm that was not subjected expression of a recombinant DNA methyltransferase. In other embodiments, an outcross of an individual line exhibiting discrete variability can be to a plant that has not been subjected to expression of a recombinant DNA methyltransferase but is not isogenic to the individual line exhibiting discrete variation. In other embodiments, an outcross of an individual line exhibiting discrete variability can be to a plant that has been subjected to expression of a recombinant DNA methyltransferase but is isogenic or is not isogenic to the individual line exhibiting discrete variation. Thus, in certain embodiments, an outcross of an individual line exhibiting discrete variability can also be to a plant that comprises one or more chromosomal or epigenetic polymorphisms that do not occur in the individual line exhibiting discrete variability, to a plant derived from partially or wholly different germplasm, or to a plant of a different heterotic group (in instances where such distinct heterotic groups exist). It is also recognized that such an outcross can be made in either direction. Thus, an individual line exhibiting discrete variability can be used as either a pollen donor or a pollen recipient to a plant that has not been subjected to expression of a recombinant DNA methyltransferase in such outcrosses. In certain embodiments, the progeny of the outcross are then selfed to establish individual lines that can be separately screened to identify lines with improved traits relative to parental lines. Such individual lines that exhibit the improved traits are then selected and can be propagated by further selfing

In certain embodiments, sub-populations of plants comprising the useful traits and epigenetic changes induced by expression of a recombinant DNA methyltransferase can be selected and bred as a population. Such populations can then be subjected to one or more additional rounds of selection for the useful traits and/or epigenetic changes to obtain subsequent sub-populations of plants exhibiting the useful trait and/or epigenetic changes. Any of these sub-populations can also be used to generate a seed lot. In an exemplary embodiment, plants subjected to expression of a recombinant DNA methyltransferase and exhibiting a Msh1-dr phenotype can be selfed or outcrossed to obtain an F1 generation. A bulk selection at the F1, F2, and/or F3 generation can thus provide a population of plants exhibiting the useful trait and/or epigenetic changes and/or a seed lot. In certain embodiments, it is also anticipated that populations of progeny plants or progeny seed lots comprising a mixture of inbred and/or hybrid germplasms can be derived from populations comprising hybrid germplasm (i.e. plants arising from cross of one inbred line to a distinct inbred line). Seed lots thus obtained from these exemplary method or other methods provided herein can comprise seed wherein at least 25%-50%, 50%-70%, 70%-80%, 80%-90%, 90%-95%, or 95%-100% of progeny plants grown from the seed exhibit a useful trait to a greater extent than control plants. The selection would provide the most robust and vigorous of the population for seed lot production. Seed lots produced in this manner could be used for either breeding or sale. In certain embodiments, a seed lot comprising seed wherein at least 25%-50%, 50%-70%, 70%-80%, 80%-90%, 90%-95%, or 95%-100% of progeny plants grown from the seed exhibit a useful trait associated with one or more epigenetic changes, wherein the epigenetic changes are associated with CG hyper-methylation and/or CHG and/or CHH hyper-methylation at one or more nuclear chromosomal loci, preferably including, but not limited to, pericentromeric regions and transposable elements, in comparison to a control plant that does not exhibit the useful trait, and wherein the seed or progeny plants grown from said seed that is epigenetically heterogenous are obtained. A seed lot obtainable by these methods can include at least 1-100, 100-500, 500-1000, 1000-5000, 5,000-10,000, 10,000-1,000,000 or more seeds.

Altered chromosomal loci that can confer at least one useful trait can also be identified and selected by performing appropriate comparative analyses of reference plants that do not exhibit the useful traits and test plants obtained from a parental plant or plant cell that had been subjected to expression of a recombinant DNA methyltransferase and obtaining either the altered loci or plants comprising the altered loci. It is anticipated that a variety of reference plants and test plants can be used in such comparisons and selections. In certain embodiments, the reference plants that do not exhibit the useful trait include, but are not limited to, any of: a) a wild-type plant; b) a distinct subpopulation of plants within a given F2 population of plants of a given plant line (where the F2 population is any applicable plant type or variety); c) an F1 population exhibiting a wild type phenotype (where the F1 population is any applicable plant type or variety); and/or, d) a plant that is isogenic to the parent plants or parental cells of the test plants prior to expression of a recombinant DNA methyltransferase in those parental plants or plant cells (i.e. the reference plant is isogenic to the plants or plant cells that were later subjected to expression of a recombinant DNA methyltransferase to obtain the test plants). In certain embodiments, the test plants that exhibit the useful trait include, but are not limited to, any of: a) any non-transgenic segregants that exhibit the useful trait and that were derived from parental plants or plant cells that had been subjected to expression of a recombinant DNA methyltransferase, b) a distinct subpopulation of plants within a given F2 population of plants of a given plant line that exhibit the useful trait (where the F2 population is any applicable plant type or variety); (c) any progeny plants obtained from the plants of (a) or (b) that exhibit the useful trait; or d) a plant or plant cell that had been subjected to expression of a recombinant DNA methyltransferase that exhibit the useful trait.

In general, another objective of these comparisons is to identify differences in the small RNA profiles and/or methylation of certain chromosomal DNA loci between test plants that exhibit the useful traits and reference plants that do not exhibit the useful traits. Altered chromosomal loci thus identified can then be isolated or selected, in plants to obtain plants exhibiting the useful traits or for breeding the plants to obtain progeny with improvements in the useful traits.

In certain embodiments, altered chromosomal loci can be identified by identifying small RNAs that are up or down regulated in the test plants (in comparison to reference plants). This method is based in part on identification of altered chromosomal loci where small interfering RNAs direct the methylation of specific gene targets by RNA-directed DNA methylation (RdDM). The RNA-directed DNA methylation (RdDM) process has been described (Chinnusamy V et al. Sci China Ser C-Life Sci. (2009) 52(4): 331-343). Any applicable technology platform can be used to compare small RNAs in the test and reference plants, including, but not limited to, microarray-based methods (Franco-Zorilla et al. Plant J. 2009 59(5):840-50), deep sequencing based methods (Wang et al. The Plant Cell 21:1053-1069 (2009)), and the like. Any applicable technology platform can be used to compare small RNAs in the test and reference plants, including, but not limited to: microarray-based methods (Franco-Zorilla et al. Plant J. 200959(5):840-50); deep sequencing based methods (Wang et al. The Plant Cell 21:1053-1069(2009); Wei et al., Proc Natl Acad Sci USA. 2014 Feb. 19, 111(10): 3877-3882; Zhai et al., Methods. 2013 Jun. 28. pii: S1046-2023(13)00237-5. doi: 10.1016/j.ymeth.2013.06.025 or J. Zhai et al., Methods (2013), http://dx.doi.org/10.1016/j.ymeth.2013.06.025); U.S. Pat. No., 7,550,583; U.S. Pat. No. 8,399,221; U.S. Pat. No. 8,399,222; U.S. Pat. No. 8,404,439; U.S. Pat. No. 8,637,276; Rosas-Cárdenas et al., (2011) Plant Methods 2011, 7:4; Moyano et al., BMC Genomics. 2013 Oct. 11; 14:701; Eldem et al., PLoS One. 2012; 7(12):e50298; Barber et al., Proc Natl Acad Sci USA. 2012 Jun. 26; 109(26):10444-9; Gommans et al., Methods Mol Biol. 2012; 786:167-78; and the like.

DNA methylation and sRNAs corresponding to these regions can change in progeny plants when two parent plants are crossed. Tomato progeny plants from a cross displayed transgressive sRNAs that were more abundant in the progeny than in either parent (Shivaprasad et al., EMBO J. 2012 Jan. 18; 31(2):257-66). A cross between two maize lines, B73 and Mo17, yielded paramutation type switches of the DNA methylation pattern of one parent chromosome being switched to that of the other parental chromosome at the corresponding loci (Regulski et al., Genome Res. 2013 October; 23(10):1651-62). A cross between Arabidopsis plants produced progeny wherein the DNA methylation patterns of one parental chromosome were imposed onto the other parental chromosome, either gaining or losing DNA methylation levels (Greaves et al., Proc Natl Acad Sci USA. 2014 Feb. 4; 111(5):2017-22). These non-limiting examples indicate DNA methylation patterns can be more complex than just additive patterns from both parents. Accordingly, an objective is to identify new combinations of altered chromosomal loci in progeny plants that have new patterns of DNA methylation and/or of sRNA profiles. New combinations of altered chromosomal loci can result both from genetic segregation of altered chromosomal loci in the progeny as well as due to changes in DNA methylation and sRNA profiles due to transgressive, paramutation type switching, and other biological processes. In certain embodiments, altered chromosomal loci are derived from a parental plant subjected to expression of a recombinant DNA methyltransferase. In certain embodiments, altered chromosomal loci are derived from the formation of new patterns of DNA methylation and sRNA levels from the interaction of altered chromosomal loci derived from a parental plant subjected to expression of a recombinant DNA methyltransferase with chromosomal loci from a second plant. Said second plant can be from a parental plant subjected to suppression of MSH1 or expression of a recombinant DNA methyltransferase or from a parental plant not subjected to suppression of MSH1 or expression of a recombinant DNA methyltransferase. In certain embodiments, crossing parental lines both previously subjected to expression of a recombinant DNA methyltransferase and containing different groupings of altered chromosomal loci provides a method of creating new combinations of altered chromosomal loci.

In certain embodiments, altered chromosomal loci can be identified by identifying histone proteins associated with a locus and that are methylated or acylated in the test plants (in comparison to reference plants). The analysis of chromosomal loci associated with methylated or acylated histones can be accomplished by enriching and sequencing those loci using antibodies that recognize methylated or acylated histones. Identification of chromosomal regions associated with methylation or acetylation of specific lysine residues of histone H3 by using antibodies specific for H3K4me3, H3K9ac, H3K27me3, and H3K36me3 has been described (Li et al., Plant Cell 20:259-276, 2008; Wang et al. The Plant Cell 21:1053-1069 (2009).

In certain embodiments, one or more altered chromosomal loci can be identified by identifying chromosomal regions (genomic DNA) that has an altered methylation status in the test plants (in comparison to reference plants, see U.S. Provisional Applications 61/970,424 and 61/863,267, incorporated herein by reference in their entirety). An altered methylation status can comprise either the presence or absence of methylation in one or more chromosomal loci of a test plant in comparison to a reference plant. Any applicable technology platform can be used to compare the methylation status of chromosomal loci in the test and reference plants. Applicable technologies for identifying chromosomal loci with changes in their methylation status include, but not limited to, methods based on immunoprecipitation of DNA with antibodies that recognize 5-methylcytidine, methods based on use of methylation dependent restriction endonucleases and PCR such as McrBC-PCR methods (Rabinowicz, et al. Genome Res. 13: 2658-2664 2003; Li et al., Plant Cell 20:259-276, 2008), sequencing of bisulfite-converted DNA (Frommer et al. Proc. Natl. Acad. Sci. U.S.A. 89 (5): 1827-31; Tost et al. BioTechniques 35 (1): 152-156, 2003), methylation-specific PCR analysis of bisulfite treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415(2):145-50, 2011), single molecule real time sequencing methods (Flusberg et al. Nature Methods 7, 461-465), high resolution melting analysis (Wojdacz and Dobrovic (2007) Nucleic Acids Res. 35 (6): e41), and the like.

Additional applicable technologies for identifying chromosomal loci with changes in their DNA methylation status include, but not limited to, the preparation, amplification and analysis of Methylome libraries as described in U.S. Pat. No. 8,440,404; using Methylation-specific binding proteins as described in U.S. Pat. No. 8,394,585; determining the average DNA methylation density of a locus of interest within a population of DNA fragments as described in U.S. Pat. No. 8,361,719; by methylation-sensitive single nucleotide primer extension (Ms-SNuPE), for determination of strand-specific methylation status at cytosine residues as described in U.S. Pat. No. 7,037,650; a method for detecting a methylated CpG-containing nucleic acid present in a specimen by contacting the specimen with an agent that modifies unmethylated cytosine and amplifying the CpG-containing nucleic acid using CpG-specific oligonucleotide primers as described in U.S. Pat. No. 6,265,171; an improved method for the bisulfite conversion of DNA for subsequent analysis of DNA methylation as described in U.S. Pat. No. 8,586,302; for treating genomic DNA samples with sodium bisulfite to create methylation-dependent sequence differences, followed by detection with fluorescence-based quantitative PCR techniques as described in U.S. Pat. No. 8,323,890; a method for retaining methylation pattern in globally amplified DNA as described in U.S. Pat. No. 7,820,385; a method for detecting cytosine methylations DNA as described in U.S. Pat. No. 8,241,855; a method for quantification of methylated DNA as described in U.S. Pat. No. 7,972,784; a highly sensitive method for the detection of cytosine methylation patterns as described in U.S. Pat. No. 7,229,759; additional methods for detecting DNA methylation changes are described in U.S. Pat. No. 7,943,308 and U.S. Pat. No. 8,273,528.

Methods for introducing various chromosomal modifications that can confer a useful trait into a plant, as well as the plants, plant parts, and products of those plant parts are also provided herein. Chromosomal alterations and/or chromosomal mutations induced by expression of a recombinant DNA methyltransferase can be identified as described herein. Once identified, chromosomal modifications including, but not limited to, chromosomal alterations, chromosomal mutations, or transgenes that provide for the same genetic effect as the chromosomal alterations and/or chromosomal mutations induced by expression of a recombinant DNA methyltransferase can be introduced into host plants to obtain plants that exhibit the desired trait. In this context, the “same genetic effect” means that the introduced chromosomal modification provides for an increase and/or a reduction in expression of one or more endogenous plant genes that is similar to that observed in a plant that has been subjected to expression of a recombinant DNA methyltransferase and exhibits the useful trait. In certain embodiments where an endogenous gene is methylated in a plant subjected to expression of a recombinant DNA methyltransferase and exhibits both reduced expression of that gene and a useful trait, chromosomal modifications in other plants that also result in reduced expression of that gene and the useful trait are provided. In certain embodiments where an endogenous gene is demethylated in a plant subjected to expression of a recombinant DNA methyltransferase and exhibits both increased expression of that gene and a useful trait, chromosomal modifications in other plants that also result in increased expression of that gene and that useful trait are provided.

In certain embodiments, the chromosomal modification that is introduced is one or more altered chromosomal loci. Altered chromosomal loci including, but not limited to, a difference in a methylation state can be introduced by crossing a plant comprising the altered chromosomal loci to a plant that lacks the altered chromosomal loci and selecting for the presence of the alteration in F1, F2, or any subsequent generation progeny plants of the cross. In still other embodiments, the altered chromosomal loci in specific target genes can be introduced by expression of a siRNA or hairpin RNA or Pol IV/Pol V combination (Johnson et al., Nature. 2014 Mar. 6; 507(7490):124-8) targeted to that gene by RNA directed DNA methylation (Chinnusamy V et al. Sci China Ser C-Life Sci. (2009) 52(4): 331-343; Cigan et al. Plant J 43 929-940, 2005; Heilersig et al. (2006) Mol Genet Genomics 275 437-449; Miki and Shimamoto, Plant Journal 56(4):539-49; Okano et al. Plant Journal 53(1):65-77, 2008).

In certain embodiments, the chromosomal modification is a chromosomal mutation. Chromosomal mutations that provide for reductions or increases in expression of an endogenous gene of a chromosomal locus can include, but are not limited to, insertions, deletions, and/or substitutions of nucleotide sequences in a gene. Chromosomal mutations can result in decreased expression of a gene by a variety of mechanisms that include, but are not limited to, introduction of missense codons, frame-shift mutations, premature translational stop codons, promoter deletions, mutations that disrupt mRNA processing, and the like. Chromosomal mutations that result in increased expression of a gene include, but are not limited to, promoter substitutions, removal of negative regulatory elements from the gene, and the like. Chromosomal mutations can be introduced into specific loci of a plant by any applicable method. Applicable methods for introducing chromosomal mutations in endogenous plant chromosomal loci include, but are not limited to, homologous double stranded break repair (Wright et al., Plant J. 44, 693, 2005; D′Halluin, et al., Plant Biotech. J. 6:93, 2008), non-homologous end joining or a combination of non-homologous end joining and homologous recombination (reviewed in Puchta, J. Exp. Bot. 56, 1, 2005; Wright et al., Plant J. 44, 693, 2005), meganuclease-induced, site specific double stranded break repair (WO/06097853A1, WO/06097784A1, WO/04067736A2, U.S. 20070117128A1), and zinc finger nuclease mediated homologous recombination (WO 03/080809, WO 05/014791, WO 07014275, WO 08/021207). In still other embodiments, desired mutations in endogenous plant chromosomal loci can be identified through use of the TILLING technology (Targeting Induced Local Lesions in Genomes) as described (Henikoff et al., Plant Physiol. 2004, 135:630-636). In still other embodiments, CRISPR/CAS9 systems are used for genome editing to create mutations or gene replacement and modifications or alterations (Strauβ and Lahaye, Mol Plant. 2013 September; 6(5):1384-7).

In other embodiments, chromosomal modifications that provide for the desired genetic effect can comprise a transgene. Transgenes that can result in decreased expression of an gene by a variety of mechanisms that include, but are not limited to, dominant-negative mutants, a small inhibitory RNA (siRNA), a microRNA (miRNA), a co-suppressing sense RNA, and/or an anti-sense RNA and the like. US patents incorporated herein by reference in their entireties that describe suppression of endogenous plant genes by transgenes include U.S. Pat. No. 7,109,393, U.S. Pat. No. 5,231,020 and U.S. Pat. No. 5,283,184 (co-suppression methods); and U.S. Pat. No. 5,107,065 and U.S. Pat. No. 5,759,829 (antisense methods). In certain embodiments, transgenes specifically designed to produce double-stranded RNA (dsRNA) molecules with homology to the endogenous gene of a chromosomal locus can be used to decrease expression of that endogenous gene. In such embodiments, the sense strand sequences of the dsRNA can be separated from the antisense sequences by a spacer sequence, preferably one that promotes the formation of a dsRNA (double-stranded RNA) molecule. Examples of such spacer sequences include, but are not limited to, those set forth in Wesley et al., Plant J., 27(6):581-90 (2001), and Hamilton et al., Plant J., 15:737-746 (1998). Vectors for inhibiting endogenous plant genes with transgene-mediated expression of hairpin RNAs are disclosed in U.S. Patent Application Nos. 20050164394, 20050160490, and 20040231016, each of which is incorporated herein by reference in their entirety.

Transgenes that result in increased expression of a gene of a chromosomal locus include, but are not limited to, a recombinant gene fused to heterologous promoters that are stronger than the native promoter, a recombinant gene comprising elements such as heterologous introns, 5′ untranslated regions, 3′ untranslated regions that provide for increased expression, and combinations thereof. Such promoter, intron, 5′ untranslated, 3′ untranslated regions, and any necessary polyadenylation regions can be operably linked to the DNA of interest in recombinant DNA molecules that comprise parts of transgenes useful for making chromosomal modifications as provided herein.

Exemplary promoters useful for expression of transgenes, including expression of a recombinant DNA methyltransferase, include, but are not limited to, enhanced or duplicate versions of the viral CaMV35S and FMV35S promoters (U.S. Pat. No. 5,378,619), the cauliflower mosaic virus (CaMV) 19S promoters, the rice Act1 promoter and the Figwort Mosaic Virus (FMV) 35S promoter (U.S. Pat. No. 5,463,175). Exemplary introns useful for transgene expression include, but are not limited to, the maize hsp70 intron (U.S. Pat. No. 5,424,412), the rice Act1 intron (McElroy et al., 1990, The Plant Cell, Vol. 2, 163-171), the CAT-1 intron (Cazzonnelli and Velten, Plant Molecular Biology Reporter 21: 271-280, September 2003), the pKANNIBAL intron (Wesley et al., Plant J. 2001 27(6):581-90; Collier et al., 2005, Plant J 43: 449-457), the PIV2 intron (Mankin et al. (1997) Plant Mol. Biol. Rep. 15(2): 186-196) and the “Super Ubiquitin” intron (U.S. Pat. No. 6,596,925; Collier et al., 2005, Plant J 43: 449-457). Exemplary 3′ polyadenylation sequences include, but are not limited to, and Agrobacterium tumor-inducing (Ti) plasmid nopaline synthase (NOS) gene; the CaMV 35S 3′ polyadenylation region, the OCS polyadenylation region, and the pea ssRUBISCO E9 gene polyadenylation sequences.

Plant lines and plant populations obtained by the methods provided herein can be screened and selected for a variety of useful traits by using a wide variety of techniques. In particular embodiments provided herein, individual progeny plant lines or populations of plants obtained from the selfs or outcrosses of plants subjected to expression of a recombinant DNA methyltransferase to other plants are screened and selected for the desired useful traits. In certain embodiments, the screened and selected trait is improved plant yield. In certain embodiments, such yield improvements are improvements in the yield of a plant line relative to one or more parental line(s) under non-stress conditions. Non-stress conditions comprise conditions where water, temperature, nutrients, minerals, and light fall within typical ranges for cultivation of the plant species. Such typical ranges for cultivation comprise amounts or values of water, temperature, nutrients, minerals, and/or light that are neither insufficient nor excessive. In certain embodiments, such yield improvements are improvements in the yield of a plant line relative to parental line(s) under abiotic stress conditions. Such abiotic stress conditions include, but are not limited to, conditions where water, temperature, nutrients, minerals, and/or light that are either insufficient or excessive. Abiotic stress conditions would thus include, but are not limited to, drought stress, osmotic stress, nitrogen stress, phosphorous stress, mineral stress, heat stress, cold stress, and/or light stress. In this context, mineral stress includes, but is not limited to, stress due to insufficient or excessive potassium, calcium, magnesium, iron, manganese, copper, zinc, boron, aluminum, or silicon. In this context, mineral stress includes, but is not limited to, stress due to excessive amounts of heavy metals including, but not limited to, cadmium, copper, nickel, zinc, lead, and chromium.

Improvements in yield in plant lines obtained by the methods provided herein can be identified by direct measurements of wet or dry biomass including, but not limited to, grain, lint, leaves, stems, or seed. Improvements in yield can also be assessed by measuring yield related traits that include, but are not limited to, 100 seed weight, a harvest index, and seed weight. In certain embodiments, such yield improvements are improvements in the yield of a plant line relative to one or more parental line(s) and can be readily determined by growing plant lines obtained by the methods provided herein in parallel with the parental plants. In certain embodiments, field trials to determine differences in yield whereby plots of test and control plants are replicated, randomized, and controlled for variation can be employed (Giesbrecht F G and Gumpertz M L. 2004. Planning, Construction, and Statistical Analysis of Comparative Experiments. Wiley. New York; Mead, R. 1997. Design of plant breeding trials. In Statistical Methods for Plant Variety Evaluation. eds. Kempton and Fox. Chapman and Hall. London.). Methods for spacing of the test plants (i.e. plants obtained with the methods of this invention) with check plants (parental or other controls) to obtain yield data suitable for comparisons are provided in references that include, but are not limited to, any of Cullis, B. et al. J. Agric. Biol. Env. Stat. 11:381-393; and Besag, J. and Kempton, R A. 1986. Biometrics 42: 231-251.).

In certain embodiments, the screened and selected trait is improved resistance to biotic plant stress relative to the parental lines. Biotic plant stress includes, but is not limited to, stress imposed by plant fungal pathogens, plant bacterial pathogens, plant viral pathogens, insects, nematodes, and herbivores. In certain embodiments, screening and selection of plant lines that exhibit resistance to fungal pathogens including, but not limited to, an Alternaria sp., an Ascochyta sp., a Botrytis sp.; a Cercospora sp., a Colletotrichum sp., a Diaporthe sp., a Diplodia sp., an Erysiphe sp., a Fusarium sp., Gaeumanomyces sp., Helminthosporium sp., Macrophomina sp., a Nectria sp., a Peronospora sp., a Phakopsora sp., Phialophora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncinula sp, a Venturia sp., and a Verticillium sp. is provided. In certain embodiments, screening and selection of plant lines that exhibit resistance to bacterial pathogens including, but not limited to, an Erwinia sp., a Pseudomonas sp., and a Xanthamonas sp. is provided. In certain embodiments, screening and selection of plant lines that exhibit resistance to insects including, but not limited to, aphids and other piercing/sucking insects such as Lygus sp., lepidoteran insects such as Armigera sp., Helicoverpa sp., Heliothis sp., and Pseudoplusia sp., and coleopteran insects such as Diabroticus sp. is provided. In certain embodiments, screening and selection of plant lines that exhibit resistance to nematodes including, but not limited to, Meloidogyne sp., Heterodera sp., Belonolaimus sp., Ditylenchus sp., Globodera sp., Naccobbus sp., and Xiphinema sp. is provided.

Other useful traits that can be obtained by the methods provided herein include various seed quality traits including, but not limited to, improvements in either the compositions or amounts of oil, protein, or starch in the seed. Still other useful traits that can be obtained by methods provided herein include, but are not limited to, increased biomass, non-flowering, male sterility, digestability, seed filling period, maturity (either earlier or later as desired), reduced lodging, and plant height (either increased or decreased as desired).

In addition to any of the aforementioned traits, particularly useful traits that can be obtained by the methods provided herein also include, but are not limited to: i) agronomic traits (flowering time, days to flower, days to flower-post rainy, days to flowering; ii) fungal disease resistance; iii) grain related traits: (Grain dry weight, grain number, grain number per square meter, Grain weight over panicle, seed color, seed luster, seed size); iv) growth and development stage related traits (basal tillers number, days to harvest, days to maturity, nodal tillering, plant height, plant height); v) infloresence anatomy and morphology trait (threshability); vi) Insect damage resistance; vii) leaf related traits (leaf color, leaf midrib color, leaf vein color, flag leaf weight, leaf weight, rest of leaves weight); viii) mineral and ion content related traits (shoot potassium content, shoot sodium content); ix) panicle, pod, or ear related traits (number of panicles and seeds, harvest index, panicle weight); x) phytochemical compound content (plant pigmentation); xii) spikelet anatomy and morphology traits (glume color, glume covering); xiii) stem related trait (stem over leaf weight, stem weight); and xiv) miscellaneous traits (stover related traits, metabolised energy, nitrogen digestibility, organic matter digestibility, stover dry weight).

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena; Hordeum, Secale, and Triticum.

In some embodiments, plants or plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago s sp.), orchard grass, tall fescue, perennial ryegrass, creeping bent grass, and redtop.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Constitutive Expression of Soybean DRM2 in Soybean

The binary vector for plant transformation is pCAMBIA1300-BAR, a pCAMBIA1300 derived vector that is modified to replace the hygromycin selectable marker with a Streptomyces hygroscopicus bar gene for selection of transformed plant cells with bialophos or phosphinothricin. The BAR gene is commercially synthesized with flanking XhoI sites (SEQ ID NO: 1), digested with XhoI, purified, and ligated into pCAMBIA1300 restricted with XhoI to remove the hygromycin gene. The resulting pCAMBIA1300-BAR binary plasmid has the BAR selectable gene as a CaMV35S promoter/BAR/CaMV 35S terminator (polyadenylation site) cassette for use as a selectable marker in plants.

A 433 bp CaMV 35S promoter is commercially synthesized to have 5′ BamHI and 3′ XhoI restriction sites (SEQ ID NO: 3). A modified GFP (CLOVER) synthetic gene with a double nuclear localization signal (2×NLS) is commercially synthesized to lack a stop codon and to have a 5′ XhoI site and 3′ SacII and KpnI sites (SEQ ID NO: 4). This 2×NLS-GFP DNA region is useful for creating protein fusions to the N-terminus of proteins for visualizing their expression. After restriction with BamHI and XhoI enzymes of the CaMV 35S 433 bp promoter fragment (BamHI/CaMV 35S Pro/XhoI); and XhoI and KpnI restriction digestion of the 2×NLS-GFP DNA (XhoI/2×NLS-GFP DNA/KpnI); and BamHI and KpnI restriction digestion of pUC19; the DNA fragments are electrophoresed on an agarose gel, recovered by Qiagen column purification, and ligated in a 3 pc ligation, and transformed into E. coli. The resulting plasmid is pUC19:BamHI/CaMV Pro/2×NLS-GFP/SacII/KpnI).

A synthetic soybean DRM2 coding region attached to a NOS 3′ polyadenylation site, with a 5′ SacII and a 3′ SbfI restriction sites is commercially synthesized (SacII/SoyDRM2/NOS3′/SbfI, SEQ ID NO: 7). The reading frame across the SacII is inframe between the 2×NLS-GFP/SacII and the SacII/DRM2 fragment such that a fusion protein is made when these fragments are joined.

A 5′ BamHI and 3′ Sac II restriction digest of pUC19:BamHI/CaMV Pro/2×NLS-GFP/SacII/KpnI releases a BamHI/CaMV Pro/2×NLS-GFP/SacII DNA fragment; restriction digestion of the commercially synthesized SoyDRM2/NOS3′ (SEQ ID NO: 7) with SacII and SbfI; and a BamHI and SbfI restriction digestion of the plasmid vector pCAMBIA1300-BAR plasmid vector are performed, the fragments are gel purified, recovered on Qiagen DNA columns, ligated as a 3 piece DNA ligation, transformed into E. coli, and a CAMBIA1300-BAR vector containing a CaMV 35S Promoter/2×NLS-GFP-SoyDRM2/NOS3′ DNA cassette is obtained (herein named pCAMBIA1300-BAR/CaMV 35S Promoter/2×NLS-GFP-SoyDRM2/NOS3′) and transformed into Agrobacterium tumefaciens.

Transgenic Thorne soybeans plants are produced with pCAMBIA1300-BAR/CaMV 35S Promoter/2×NLS-GFP-SoyDRM2/NOS3′ in Agrobacterium tumefaciens and using glufosinate as the selection system as described (Zhang et al., Plant Cell, Tissue and Organ Culture 56: 37-46, 1999). Said transgenic soybean plants are screened for those that express the 2×NLS-GFP-DRM2 protein by fluorescence microscopy to detect GFP expression and by real time PCR analysis of cDNA made from isolated RNA from the plants to detect the 2×NLS-GFP-SoyDRM2 transcript. Transgenic soybean plants expressing 2×NLS-GFP-SoyDRM2 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their non-transgenic progeny, display enhanced DNA methylation and/or sRNAs, relative to the parental plant controls. If higher levels of DNA methylation are desired, the transgenic methyltransferase can be maintained in more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 2

### Constitutive Expression of Soybean Catalytic Domain of DRM2 in Soybean

A synthetic soybean catalytic domain of the DRM2 coding region attached to a NOS 3′ polyadenylation site, with a 5′ SacII and a 3′ SbfI restriction sites is commercially synthesized (SacII/SoycatalyticDRM2/NOS3′/SbfI, SEQ ID NO: 9). The reading frame across the SacII is inframe between the 2×NLS-GFP/SacII and the SacII/SoycatalyticDRM2 fragment such that a fusion protein is made when these fragments are joined.

A 5′ BamHI and 3′ Sac II restriction digest of pUC19:BamHI/CaMV Pro/2×NLS-GFP/SacII/KpnI releases a BamHI/CaMV Pro/2×NLS-GFP/SacII DNA fragment; restriction digestion of the commercially synthesized SoycatalyticDRM2/NOS3′ (SEQ ID NO: 9) DNA with SacII and SbfI; and a BamHI and SbfI restriction digestion of the plasmid vector pCAMBIA1300-BAR DNA are performed, the fragments are gel purified, recovered on Qiagen DNA columns, ligated as a 3 piece DNA ligation, transformed into E. coli, and a CAMBIA1300-BAR vector containing a CaMV 35S Promoter/2×NLS-GFP-SoycatalyticDRM2/NOS3′ DNA cassette is obtained (herein named pCAMBIA1300-BAR/CaMV 35S Promoter/2×NLS-GFP-SoycatalyticDRM2/NOS3′) and transformed into Agrobacterium tumefaciens.

Transgenic Thorne soybeans plants are produced with pCAMBIA1300-BAR/CaMV 35S Promoter/2×NLS-GFP-SoycatalyticDRM2/NOS3′ in Agrobacteria and using glufosinate as the selection system as described (Zhang et al., Plant Cell, Tissue and Organ Culture 56: 37-46, 1999). Said transgenic soybean plants are screened for those that express the 2×NLS-GFP-SoycatalyticDRM2 protein by fluorescence microscopy to detect GFP expression and by real time PCR analysis of cDNA made from isolated RNA from the plants to detect the 2×NLS-GFP-SoycatalyticDRM2 transcript. Transgenic soybean plants expressing 2×NLS-GFP-SoycatalyticDRM2 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their non-transgenic progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 3

### Constitutive Expression of Soybean CMT3 in Soybean

A synthetic soybean CMT3 coding region attached to a NOS 3′ polyadenylation site, with a 5′ SacII and a 3′ SbfI restriction sites is commercially synthesized (SacII/SoyCMT3/NOS3′/SbfI, SEQ ID NO: 11). The reading frame across the SacII is inframe between the 2×NLS-GFP/SacII and the SacII/SoyCMT3 fragment such that a fusion protein is made when these fragments are joined.

A 5′ BamHI and 3′ Sac II restriction digest of pUC19:BamHI/CaMV Pro/2×NLS-GFP/SacII/KpnI releases a BamHI/CaMV Pro/2×NLS-GFP/SacII DNA fragment; restriction digestion of the commercially synthesized SoyCMT3/NOS3′ (SEQ ID NO: 11) with SacII and SbfI; and a BamHI and SbfI restriction digestion of the pCAMBIA1300-BAR plasmid vector are performed, the fragments are gel purified, recovered on Qiagen DNA columns, ligated as a 3 piece DNA ligation, transformed into E. coli, and a CAMBIA1300-BAR vector containing a CaMV 35S Promoter/2×NLS-GFP-SoyCMT3/NOS3′ DNA cassette is obtained (herein named pCAMBIA1300-BAR/CaMV 35S Promoter/2×NLS-GFP-SoyCMT3/NOS3′) and transformed into Agrobacterium tumefaciens.

Transgenic Thorne soybeans plants are produced with pCAMBIA1300-BAR/CaMV 35S Promoter/2×NLS-GFP-SoyCMT3/NOS3′ in Agrobacteria and using glufosinate as the selection system as described (Zhang et-al., Plant Cell, Tissue and Organ Culture 56: 37-46, 1999). Said transgenic soybean plants are screened for those that express the 2×NLS-GFP-DRM2 protein by fluorescence microscopy to detect GFP expression and by real time PCR analysis of cDNA made from isolated RNA from the plants to detect the 2×NLS-GFP-SoyCMT3 transcript. Transgenic soybean plants expressing 2×NLS-GFP-SoyCMT3 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their non-transgenic progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 4

### Constitutive Expression of Soybean Catalytic CMT3 in Soybean

A synthetic soybean catalytic domain of CMT3 coding region attached to a NOS 3′ polyadenylation site, with a 5′ SacII and a 3′ SbfI restriction sites is commercially synthesized (SacII/SoycatalyticCMT3/NOS3′/SbfI, SEQ ID NO: 13). The reading frame across the SacII is inframe between the 2×NLS-GFP/SacII and the SacII/SoycatalyticCMT3 fragment such that a fusion protein is made when these fragments are joined.

A 5′ BamHI and 3′ Sac II restriction digest of pUC19:BamHI/CaMV Pro/2×NLS-GFP/SacII/KpnI releases a BamHI/CaMV Pro/2×NLS-GFP/SacII DNA fragment; restriction digestion of the commercially synthesized SoycatalyticCMT3/NOS3′ (SEQ ID NO: 13) with SacII and SbfI; and a BamHI and SbfI restriction digestion of the plasmid vector pCAMBIA1300-BAR plasmid vector are performed, the fragments are gel purified, recovered on Qiagen DNA columns, ligated as a 3 piece DNA ligation, transformed into E. coli, and a CAMBIA1300-BAR vector containing a CaMV 35S Promoter/2×NLS-GFP-SoycatalyticCMT3/NOS3′ DNA cassette is obtained (herein named pCAMBIA1300-BAR/CaMV 35S Promoter/2×NLS-GFP-SoycatalyticCMT3/NOS3′) and transformed into Agrobacterium tumefaciens.

Transgenic Thorne soybeans plants are produced with pCAMBIA1300-BAR/CaMV 35S Promoter/2×NLS-GFP-SoycatalyticCMT3/NOS3′ in Agrobacteria and using glufosinate as the selection system as described (Zhang et al., Plant Cell, Tissue and Organ Culture 56: 37-46, 1999). Said transgenic soybean plants are screened for those that express the 2×NLS-GFP-DRM2 protein by fluorescence microscopy to detect GFP expression and by real time PCR analysis of cDNA made from isolated RNA from the plants to detect the 2×NLS-GFP-SoycatalyticCMT3 transcript. Transgenic soybean plants expressing 2×NLS-GFP-SoycatalyticCMT3 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their non-transgenic progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 5

### Constitutive Expression of Corn DRM2 in Corn

A maize ubiquitin promoter/intron (described in U.S. Pat. No. 6,054,574) with 5′ BamHI and 3′ SalI restriction sites (SEQ ID NO: 15) is used. It is attached to the 2×NLS-GFP DNA region (SEQ ID NO: 4) of Example 1 as follows. After restriction with BamHI and SalI enzymes of the 1,571 bp maize ubiquitin promoter/intron fragment (BamHI/UBIQ Pro/SalI); and XhoI and KpnI restriction digestion of the 2×NLS-GFP DNA (XhoI/2×NLS-GFP DNA/KpnI); and BamHI and KpnI restriction digestion of pUC19; the DNA fragments are electrophoresed on an agarose gel, recovered by Qiagen column purification, and ligated in a 3 pc ligation, and transformed into E. coli. The resulting plasmid is pUC19:BamHI/UBIQ Pro/2×NLS-GFP/SacII/KpnI).

A maize DRM2 coding region attached to a NOS 3′ polyadenylation site, with 5′ SacII and 3′ SbfI restriction sites is commercially synthesized (SacII/MaizeDRM2/NOS3′/SbfI, SEQ ID NO: 16). The reading frame across the SacII site is inframe between the 2×NLS-GFP/SacII and the SacII/Maize DRM2 fragment such that a fusion protein is made when these fragments are joined. 5′ BamHI and 3′ SacII restricted pUC19:BamHI/UBIQ Pro/2×NLS-GFP/SacII/KpnI releases a BamHI/UBIQ Pro/2×NLS-GFP/SacII DNA fragment; SacII and SbfI digestion of the maize DRM2/NOS3′ DNA (SEQ ID NO: 16); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/2×NLS-GFP-MaizeDRM2/NOS3′ DNA cassette is obtained (herein named pCAMBIA1300-BAR/UBIQ Pro/2×NLS-GFP-MaizeDRM2/NOS3′).

Transgenic maize cells are produced using the bar gene of pCAMBIA1300-BAR/UBIQ Pro/2×NLS-GFP-MaizeDRM2/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the 2×NLS-GFP-Maize DRM2 protein by fluorescence microscopy to detect GFP expression and by real time PCR analysis of cDNA made from isolated RNA from the plants to detect the 2×NLS-GFP-MaizeDRM2 transcript. Transgenic maize plants expressing 2×NLS-GFP-MaizeDRM2 are self pollinated and outcrossed to their parental line (the line prior to transformation) to obtain progeny. Non-transgenic progeny of this and later generations are self pollinated to produce maize plants with enhanced yields, relative to their parental control plants. DNA methylation and/or sRNA analysis of lines containing the 2×NLS-GFP-MaizeDRM2 gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 6

### Constitutive Expression of Corn Catalytic DRM2 in Corn

A catalytic domain of a maize DRM2 coding region attached to a NOS 3′ polyadenylation site, with 5′ SacII and 3′ SbfI restriction sites is commercially synthesized (SacII/MaizecatalyticDRM2/NOS3′/SbfI, SEQ ID NO: 18). The reading frame across the SacII site is inframe between the 2×NLS-GFP/SacII and the SacII/MaizecatalyticDRM2 fragment such that a fusion protein is made when these fragments are joined. 5′ BamHI and 3′ SacII restricted pUC19:BamHI/UBIQ Pro/2×NLS-GFP/SacII/KpnI releases a BamHI/UBIQ Pro/2×NLS-GFP/SacII DNA fragment; SacII and SbfI digestion of the MaizecatalyticDRM2/NOS3′ DNA (SEQ ID NO: 18); and, BamHI and SbfI restricted pCAMBIA 1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/2×NLS-GFP-MaizecatalyticDRM2/NOS3′ DNA cassette is obtained (herein named pCAMBIA1300-BAR/UBIQ Pro/2×NLS-GFP-MaizecatalyticDRM2/NOS3′).

Transgenic maize cells are produced using the bar gene of pCAMBIA1300-BAR/UBIQ Pro/2×NLS-GFP-MaizecatalyticDRM2/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the 2×NLS-GFP-MaizecatalyticDRM2 protein by fluorescence microscopy to detect GFP expression and by real time PCR analysis of cDNA made from isolated RNA from the plants to detect the 2×NLS-GFP-MaizecatalyticDRM2 transcript. Transgenic maize plants expressing 2×NLS-GFP-MaizecatalyticDRM2 are self pollinated and outcrossed to their parental line (prior to transformation) to obtain progeny. Non-transgenic progeny of this and later generations are self pollinated to produce maize plants with enhanced yields, relative to their parental control plants. DNA methylation and/or sRNA analysis of lines containing the 2×NLS-GFP-MaizecatalyticDRM2 gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 7

### Constitutive Expression of Corn CMT3 in Corn

A maize CMT3 coding region attached to a NOS 3′ polyadenylation site, with 5′ SacII and 3′ SbfI restriction sites is commercially synthesized (SacII/MaizeCMT3/NOS3′/SbfI, SEQ ID NO: 20). The reading frame across the SacII site is inframe between the 2×NLS-GFP/SacII and the SacII/Maize CMT3 fragment such that a fusion protein is made when these fragments are joined. 5′ BamHI and 3′ SacII restricted pUC19:BamHI/UBIQ Pro/2×NLS-GFP/SacII/KpnI releases a BamHI/UBIQ Pro/2×NLS-GFP/SacII DNA fragment; SacII and SbfI digestion of the maize CMT3/NOS3′ DNA (SEQ ID NO: 20); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/2×NLS-GFP-MaizeCMT3/NOS3′ DNA cassette is obtained (herein named pCAMBIA1300-BAR/UBIQ Pro/2×NLS-GFP-MaizeCMT3/NOS3′).

Transgenic maize cells are produced using the bar gene of pCAMBIA1300-BAR/UBIQ Pro/2×NLS-GFP-MaizeCMT3/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the 2×NLS-GFP-Maize CMT3 protein by fluorescence microscopy to detect GFP expression and by, real time PCR analysis of cDNA made from isolated RNA from the plants to detect the 2×NLS-GFP-MaizeCMT3 transcript. Transgenic maize plants expressing 2×NLS-GFP-MaizeCMT3 are self pollinated and outcrossed to their parental line (prior to transformation) to obtain progeny. Non-transgenic progeny of this and later generations are self pollinated to produce maize plants with enhanced yields, relative to their parental control plants. DNA methylation and/or sRNA analysis of lines containing the 2×NLS-GFP-MaizeCMT3 gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 8

### Constitutive Expression of Corn Catalytic CMT3 in Corn

A maize CMT3 catalytic coding region attached to a NOS 3′ polyadenylation site, with 5′ SacII and 3′ SbfI restriction sites is commercially synthesized (SacII/MaizecatalyticCMT3/NOS3′/SbfI, SEQ ID NO: 22). The reading frame across the SacII site is inframe between the 2×NLS-GFP/SacII and the SacII/MaizecatalyticCMT3 fragment such that a fusion protein is made when these fragments are joined. 5′ BamHI and 3′ SacII restricted pUC19:BamHI/UBIQ Pro/2×NLS-GFP/SacII/KpnI releases a BamHI/UBIQ Pro/2×NLS-GFP/SacII DNA fragment; SacII and SbfI digestion of the MaizecatalyticCMT3/NOS3′ DNA (SEQ ID NO: 22); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/2×NLS-GFP-MaizecatalyticCMT3/NOS3′ DNA cassette is obtained (herein named pCAMBIA1300-BAR/UBIQ Pro/2×NLS-GFP-MaizecatalyticCMT3/NOS3′).

Transgenic maize cells are produced using the bar gene of pCAMBIA1300-BAR/UBIQ Pro/2×NLS-GFP-MaizecatalyticCMT3/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the 2×NLS-GFP-MaizecatalyticCMT3 protein by fluorescence microscopy to detect GFP expression and by real time PCR analysis of cDNA made from isolated RNA from the plants to detect the 2×NLS-GFP-MaizecatalyticCMT3 transcript. Transgenic maize plants expressing 2×NLS-GFP-MaizecatalyticCMT3 are self pollinated and outcrossed to their parental line (prior to transformation) to obtain progeny. Non-transgenic progeny of this and later generations are self pollinated to produce maize plants with enhanced yields, relative to their parental control plants. DNA methylation and/or sRNA analysis of lines containing the 2×NLS-GFP-MaizecatalyticCMT3 gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 9

### Constitutive Expression of Native Soybean DRM2 in Soybean

A 433 bp CaMV 35S promoter is commercially synthesized to have 5′ BamHI and 3′ SacII restriction sites (SEQ ID NO: 24). A synthetic soybean DRM2 coding region attached to a NOS 3′ polyadenylation site, with a 5′ SacII and a 3′ SbfI restriction sites is commercially synthesized (SacII/SoyDRM2/NOS3′/SbfI, SEQ ID NO: 7).

A 5′ BamHI and 3′ Sac II restriction digest of the CaMV 35S promoter (SEQ ID NO: 24) DNA; restriction digestion of the commercially synthesized SoyDRM2/NOS3′ (SEQ ID NO: 7) with SacII and SbfI; and a BamHI and SbfI restriction digestion of the plasmid vector pCAMBIA1300-BAR plasmid vector are performed, the fragments are gel purified, recovered on Qiagen DNA columns, ligated as a 3 piece DNA ligation, transformed into E. coli, and a CAMBIA1300-BAR vector containing a CaMV 35S Promoter/SoyDRM2NOS3′ DNA cassette is obtained (herein named pCAMBIA1300-BAR/CaMV 35S Promoter/SoyDRM2/NOS3′) and transformed into Agrobacterium tumefaciens.

Transgenic Thorne soybeans plants are produced with pCAMBIA1300-BAR/CaMV 35S Promoter/SoyDRM2/NOS3′ in Agrobacterium tumefaciens and using glufosinate as the selection system as described (Zhang et al., Plant Cell, Tissue and Organ Culture 56: 37-46, 1999). Said transgenic soybean plants are screened for those that express SoyDRM2 mRNA by real time PCR analysis of cDNA made from isolated RNA from the plants to detect the SoyDRM2/NOS3′ transcript. Transgenic soybean plants expressing SoyDRM2 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 10

### Constitutive Expression of Native Soybean CMT3 in Soybean

A 433 bp CaMV 35S promoter is commercially synthesized to have 5′ BamHI and 3′ SacII restriction sites (SEQ ID NO: 24). A synthetic soybean CMT3 coding region attached to a NOS 3′ polyadenylation site, with a 5′ SacII and a 3′ SbfI restriction sites is commercially synthesized (SacII/SoyCMT3/NOS3′/SbfI, SEQ ID NO: 11).

A 5′ BamHI and 3′ Sac II restriction digest of the CaMV 35S promoter (SEQ ID NO: 24) DNA; restriction digestion of the commercially synthesized SoyCMT3/NOS3′ (SEQ ID NO: 11) with SacII and SbfI; and a BamHI and SbfI restriction digestion of the plasmid vector pCAMBIA 1300-BAR plasmid vector are performed, the fragments are gel purified, recovered on Qiagen DNA columns, ligated as a 3 piece DNA ligation, transformed into E. coli, and a CAMBIA1300-BAR vector containing a CaMV 35S Promoter/SoyCMT3/NOS3′ DNA cassette is obtained (herein named pCAMBIA1300-BAR/CaMV 35S Promoter/SoyCMT3/NOS3′) and transformed into Agrobacterium tumefaciens.

Transgenic Thorne soybeans plants are produced with pCAMBIA1300-BAR/CaMV 35S Promoter/SoyCMT3/NOS3′ in Agrobacteria and using glufosinate as the selection system as described (Zhang et al., Plant Cell, Tissue and Organ Culture 56: 37-46, 1999). Said transgenic soybean plants are screened for those that express the SoyCMT3/NOS3′ mRNA by real time PCR analysis of cDNA made from isolated RNA from the plants to detect the SoyCMT3/NOS3′ transcript. Transgenic soybean plants overexpressing SoyCMT3 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 11

### Constitutive Expression of Native Corn DRM2 in Corn

A maize ubiquitin promoter/intron (described in U.S. Pat. No. 6,054,574) with 5′ BamHI and 3′ SacII restriction sites (SEQ ID NO: 25) is used. A maize DRM2 coding region attached to a NOS 3′ polyadenylation site, with 5′ SacII and 3′ SbfI restriction sites is commercially synthesized (SacII/MaizeDRM2/NOS3′/SbfI, SEQ ID NO: 16). 5′ BamHI and 3′ SacII restricted maize ubiquitin promoter/intron DNA (SEQ ID NO: 25); SacII and SbfI digestion of the maize DRM2/NOS3′ DNA (SEQ ID NO: 16); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/MaizeDRM2/NOS3′ DNA cassette is obtained (herein named pCAMBIA1300-BAR/UBIQ Pro/MaizeDRM2/NOS3′).

Transgenic maize cells are produced using the bar gene of pCAMBIA1300-BAR/UBIQ Pro/MaizeDRM2/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the Maize DRM2/NOS3′ mRNA by real time PCR analysis of cDNA made from isolated RNA from the plants to detect the MaizeDRM2/NOS3′ transcript. Transgenic maize plants overexpressing MaizeDRM2 are self pollinated and outcrossed to their parental line (the line prior to transformation) to obtain progeny. Non-transgenic progeny of this and later generations are self pollinated to produce maize plants with enhanced yields, relative to their parental control plants. DNA methylation and/or sRNA analysis of lines containing the MaizeDRM2/NOS3′ gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 12

### Constitutive Expression of Native Corn CMT3 in Corn

A maize ubiquitin promoter/intron (described in U.S. Pat. No. 6,054,574) with 5′ BamHI and 3′ SacII restriction sites (SEQ ID NO: 25) is used. A maize CMT3 coding region attached to a NOS 3′ polyadenylation site, with 5′ SacII and 3′ SbfI restriction sites is commercially synthesized (SacII/MaizeCMT3/NOS3′/SbfI, SEQ ID NO: 20). 5′ BamHI and 3′ SacII restricted maize ubiquitin promoter/intron DNA (SEQ ID NO: 25); SacII and SbfI digestion of the maize CMT3/NOS3′ DNA (SEQ ID NO: 20); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/MaizeCMT3/NOS3′ DNA cassette is obtained (herein named pCAMBIA1300-BAR/UBIQ Pro/MaizeCMT3/NOS3′).

Transgenic maize cells are produced using the bar gene of pCAMBIA1300-BAR/UBIQ Pro/MaizeCMT3/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the Maize CMT3/NOS3′ mRNA by real time PCR analysis of cDNA made from isolated RNA from the plants to detect the Maize CMT3/NOS3′ transcript. Transgenic maize plants overexpressing MaizeCMT3 are self pollinated and outcrossed to their parental line (the line prior to transformation) to obtain progeny. Non-transgenic progeny of this and later generations are self pollinated to produce maize plants with enhanced yields, relative to their parental control plants. DNA methylation and/or sRNA analysis of lines containing the MaizeCMT3/NOS3′ gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 13

### Constitutive Expression of KYP-Catalytic Domain of Soybean DRM2 in Soybean

A 433 bp CaMV 35S promoter is commercially synthesized to have 5′ BamHI and 3′ XhoI restriction sites (SEQ ID NO: 3). An Arabidopsis KYPTONITE (KYP) synthetic gene with a double nuclear localization signal (2×NLS-KYP) is commercially synthesized to lack a stop codon and to have a 5′ XhoI site and 3′ SacII and KpnI sites (SEQ ID NO: 26). After restriction with BamHI and XhoI enzymes of the CaMV 35S 433 bp promoter fragment (BamHI/CaMV 35S Pro/XhoI); and XhoI and KpnI restriction digestion of the 2×NLS-KYP DNA (SEQ ID NO: 26: XhoI/2×NLS-KYP/Kpne; and BamHI and KpnI restriction digestion of pUC19; the DNA fragments are electrophoresed on an agarose gel, recovered by Qiagen column purification, and ligated in a 3 pc ligation, and transformed into E. coli. The resulting plasmid is pUC19:BamHI/CaMV Pro/2×NLS-KYP/SacII/KpnI).

A synthetic soybean catalytic domain of the DRM2 coding region attached to a NOS 3′ polyadenylation site, with a 5′ SacII and a 3′ SbfI restriction sites is commercially synthesized (SacII/SoycatalyticDRM2/NOS3′/SbfI, SEQ ID NO: 9).

A 5′ BamHI and 3′ Sac II restriction digest of pUC19:BamHI/CaMV Pro/2×NLS-KYP/SacII/KpnI releases a BamHI/CaMV Pro/2×NLS-KYP/SacII DNA fragment; restriction digestion of the commercially synthesized SoycatalyticDRM2/NOS3′ (SEQ ID NO: 9) DNA with SacII and SbfI; and a BamHI and SbfI restriction digestion of the plasmid vector pCAMBIA1300-BAR DNA are performed, the fragments are gel purified, recovered on Qiagen DNA columns, ligated as a 3 piece DNA ligation, transformed into E. coli, and a CAMBIA1300-BAR vector containing a CaMV 35S Promoter/2×NLS-KYP-SoycatalyticDRM2/NOS3′ DNA cassette is obtained (herein named pCAMBIA1300-BAR/CaMV 35S Promoter/2×NLS-KYP-SoycatalyticDRM2/NOS3′) and transformed into Agrobacterium tumefaciens.

Transgenic Thome soybeans plants are produced with pCAMBIA1300-BAR/CaMV 35S Promoter/2×NLS-KYP-SoycatalyticDRM2/NOS3′ in Agrobacteria and using glufosinate as the selection system as described (Zhang et al., Plant Cell, Tissue and Organ Culture 56: 37-46, 1999). Said transgenic soybean plants are screened for those that express the 2×NLS-KYP-SoycatalyticDRM2 protein by real time PCR analysis of cDNA made from isolated RNA from the plants to detect the 2×NLS-KYP-SoycatalyticDRM2 transcript. Transgenic soybean plants expressing 2×NLS-KYP-SoycatalyticDRM2 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 14

### Constitutive Expression of KYP-Catalytic Domain of Soybean CMT3 in Soybean

A 433 bp CaMV 35S promoter is commercially synthesized to have 5′ BamHI and 3′ XhoI restriction sites (SEQ ID NO: 3). An Arabidopsis KYPTONITE (KYP) synthetic gene with a double nuclear localization signal (2×NLS-KYP) is commercially synthesized to lack a stop codon and to have a 5′ XhoI site and 3′ SacII and KpnI sites (SEQ ID NO: 26). After restriction with BamHI and XhoI enzymes of the CaMV 35S 433 bp promoter fragment (BamHI/CaMV 35S Pro/XhoI); and XhoI and KpnI restriction digestion of the 2×NLS-KYP DNA (SEQ ID NO: 26: XhoI/2×NLS-KYP/KpnI); and BamHI and KpnI restriction digestion of pUC19; the DNA fragments are electrophoresed on an agarose gel, recovered by Qiagen column purification, and ligated in a 3 pc ligation, and transformed into E. coli. The resulting plasmid is pUC19:BamHI/CaMV Pro/2×NLS-KYP/SacII/KpnI).

A synthetic soybean catalytic domain of the CMT3 coding region attached to a NOS 3′ polyadenylation site, with a 5′ SacII and a 3′ SbfI restriction sites is commercially synthesized (SacII/SoycatalyticCMT3/NOS3′/SbfI, SEQ ID NO: 13).

A 5′ BamHI and 3′ Sac II restriction digest of pUC19:BamHI/CaMV Pro/2×NLS-KYP/SacII/KpnI releases a BamHI/CaMV Pro/2×NLS-KYP/SacII DNA fragment; restriction digestion of the commercially synthesized SoycatalyticCMT3/NOS3′ (SEQ ID NO: 13) DNA with SacII and SbfI; and a BamHI and SbfI restriction digestion of the plasmid vector pCAMBIA1300-BAR DNA are performed, the fragments are gel purified, recovered on Qiagen DNA columns, ligated as a 3 piece DNA ligation, transformed into E. coli, and a CAMBIA1300-BAR vector containing a CaMV 35S Promoter/2×NLS-KYP-SoycatalyticCMT3/NOS3′ DNA cassette is obtained (herein named pCAMBIA1300-BAR/CaMV 35S Promoter/2×NLS-KYP-SoycatalyticCMT3/NOS3′) and transformed into Agrobacterium tumefaciens.

Transgenic Thorne soybeans plants are produced with pCAMBIA1300-BAR/CaMV 35S Promoter/2×NLS-KYP-SoycatalyticCMT3/NOS3′ in Agrobacteria and using glufosinate as the selection system as described (Zhang et al., Plant Cell, Tissue and Organ Culture 56: 37-46, 1999). Said transgenic soybean plants are screened for those that express the 2×NLS-KYP-SoycatalyticCMT3 protein by real time PCR analysis of cDNA made from isolated RNA from the plants to detect the 2×NLS-KYP-SoycatalyticCMT3 transcript. Transgenic soybean plants expressing 2×NLS-KYP-SoycatalyticCMT3 are self pollinated and outcrossed to a Thorne parental line to obtain progeny. Non-transgenic progeny of this and later generations are self pollinated to produce soybean plants with enhanced yields, relative to their parental control plants. DNA methylation and/or sRNA analysis of lines containing the transgene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 15

### Constitutive Expression of KYP-Catalytic Domain of Corn CMT3 in Corn

A maize ubiquitin promoter/intron (described in U.S. Pat. No. 6,054,574) with 5′ BamHI and 3′ SalI restriction sites (SEQ ID NO: 15) is used. An Arabidopsis KYPTONITE (KYP) synthetic gene with a double nuclear localization signal (2×NLS-synKYP) and using maize preferred codons is commercially synthesized to lack a stop codon and to have a 5′ XhoI site and 3′ SacII and KpnI sites (SEQ ID NO: 29). After restriction with BamHI and SalI enzymes of the maize ubiquitin promoter/intron (SEQ ID NO: 15); and XhoI and KpnI restriction digestion of the 2×NLS-synKYP DNA (SEQ ID NO: 29: XhoI/2×NLS-synKYP/KpnI); and BamHI and KpnI restriction digestion of pUC19; the DNA fragments are electrophoresed on an agarose gel, recovered by Qiagen column purification, and ligated in a 3 pc ligation, and transformed into E. coli. The resulting plasmid is pUC19:BamHI/UBIQ Pro/2×NLS-synKYP/SacII/KpnI).

A maize CMT3 catalytic coding region attached to a NOS 3′ polyadenylation site, with 5′ SacII and 3′ SbfI restriction sites is commercially synthesized (SacII/MaizecatalyticCMT3/NOS3′/SbfI, SEQ ID NO: 22). The reading frame across the SacII site is inframe between the 2×NLS-synKYP/SacII and the SacII/MaizecatalyticCMT3 fragment such that a fusion protein is made when these fragments are joined. 5′ BamHI and 3′ SacII restricted pUC19:BamHI/UBIQ Pro/2×NLS-synKYP/SacII/KpnI releases a BamHI/UBIQ Pro/2×NLS-synKYP/SacII DNA fragment; SacII and SbfI digestion of the MaizecatalyticCMT3/NOS3′ DNA (SEQ ID NO: 22); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/2×NLS-synKYP-MaizecatalyticCMT3/NOS3′ DNA cassette is obtained (herein named pCAMBIA1300-BAR/UBIQ Pro/2×NLS-synKYP-MaizecatalyticCMT3/NOS3′).

Transgenic maize cells are produced using the bar gene of pCAMBIA1300-BAR/UBIQ Pro/2×NLS-synKYP-MaizecatalyticCMT3/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the 2×NLS-synKYP-MaizecatalyticCMT3/NOS3′ mRNA by real time PCR analysis of cDNA made from isolated RNA from the plants to detect the 2×NLS-synKYP-MaizecatalyticCMT3/NOS3′ transcript. Transgenic maize plants overexpressing 2×NLS-synKYP-MaizecatalyticCMT3/NOS3′ are self pollinated and outcrossed to their parental line (the line prior to transformation) to obtain progeny. Non-transgenic progeny of this and later generations are self pollinated to produce maize plants with enhanced yields, relative to their parental control plants. DNA methylation and/or sRNA analysis of lines containing the 2×NLS-synKYP-MaizecatalyticCMT3/NOS3′ transgene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 16

### Constitutive Expression of KYP-Catalytic Domain of Corn DRM2 in Corn

A maize DRM2 catalytic coding region attached to a NOS 3′ polyadenylation site, with 5′ SacII and 3′ SbfI restriction sites is commercially synthesized (SacII/MaizecatalyticDRM2/NOS3′/SbfI, SEQ ID NO: 18). The reading frame across the SacII site is inframe between the 2×NLS-synKYP/SacII and the SacII/MaizecatalyticDRM2 fragment such that a fusion protein is made when these fragments are joined. 5′ BamHI and 3′ SacII restricted pUC19:BamHI/UBIQ Pro/2×NLS-synKYP/SacII/KpnI releases a BamHI/UBIQ Pro/2×NLS-synKYP/SacII DNA fragment; SacII and SbfI digestion of the MaizecatalyticDRM2/NOS3′ DNA (SEQ ID NO: 18); and, BamHI and SbfI restricted pCAMBIA1300-BAR plasmid vector DNA are gel purified, recovered on Qiagen DNA columns, ligated as a 3 piece DNA ligation, transformed into E. coli, and a CAMBIA1300-BAR vector containing a UBIQ Pro/2×NLS-synKYP-MaizecatalyticDRM2/NOS3′ DNA cassette is obtained (herein named pCAMBIA1300-BAR/UBIQ Pro/2×NLS-synKYP-MaizecatalyticDRM2/NOS3′).

Transgenic maize cells are produced using the bar gene of pCAMBIA1300-BAR/UBIQ Pro/2×NLS-synKYP-MaizecatalyticDRM2/NOS3′ as a selectable marker and bialaphos as the selective agent as described in U.S. Pat. Nos. 5,489,520 and 5,550,318 and regenerated transgenic maize plants are obtained. Said transgenic maize plants are screened for those that express the 2×NLS-synKYP-MaizecatalyticDRM2/NOS3′ mRNA by real time PCR analysis of cDNA made from isolated RNA from the plants to detect the 2×NLS-synKYP-MaizecatalyticDRM2/NOS3′ transcript. Transgenic maize plants overexpressing 2×NLS-synKYP-MaizecatalyticDRM2 are self pollinated and outcrossed to their parental line (the line prior to transformation) to obtain progeny. Non-transgenic progeny of this and later generations are self pollinated to produce maize plants with enhanced yields, relative to their parental control plants. DNA methylation and/or sRNA analysis of lines containing the 2×NLS-synKYP-MaizecatalyticDRM2/NOS3′ gene, or their progeny, display enhanced DNA methylation and/or sRNAs, relative to the parental plant controls. If higher levels of DNA methylation are desired, the transgenic methyltransferase can be maintained in one or more progeny generations prior to its removal by segregation or crossing. Highly methylated, non-transgenic lines can be used as self pollinated lines or outcrossed. Out crossed lines can be further bred or selfed to produced enhanced yield or enhanced trait lines.

### Example 17

### Identification of the Conserved Amino Acids in Plant DRM2 Proteins

A clustal omega analysis of the DRM2 protein sequences given in Table 1 was performed for the catalytic regions of these DRM2 proteins. The alignment of these 18 proteins is shown in FIG. 1. This catalytic region aligns with 322 amino acids from the Arabidopsis DRM2 catalytic domain (C-terminal) amino acids (FIG. 1). In this region there are 136 amino acids that are identical in all 18 DRM2 plant proteins in FIG. 1. This analysis provides a method for defining DRM2 group members: a clustal omega alignment of the candidate DRM2 protein to the group of 18 DRM2 proteins of Table 1 (as indicated in FIG. 1) will provide the basic alignment of the candidate protein against the DRM2 catalytic region. Candidate DRM2 proteins with at least 50% identity at the conserved amino acid positions, preferably at least 50% to 60%, 60% to 70%, 70% to 80%, 80% to 90%, 90% to 100%, or 100% are identified as DMR2 group proteins by this method. Candidate proteins with these amounts of identity at these conserved positions and which have DNA methyltransferase activity in vitro or in vivo are useful for certain embodiments of the present invention.

### Example 18

### Identification of the Conserved Amino Acids in Plant CMT3 Proteins

A clustal omega analysis of the CMT3 protein sequences given in Table 1 was performed for the catalytic regions of these CMT3 proteins. The alignment of these 16 proteins is shown in FIG. 2. This catalytic region aligns with 479 amino acids from the Arabidopsis CMT3 catalytic domain (C-terminal) amino acids (FIG. 2). In this region there are 170 amino acids that are identical in all 16 CMT3 plant proteins in FIG. 2. This analysis provides a method for defining CMT3 group members: a clustal omega alignment of the candidate CMT3 protein to the group of 16 CMT3 proteins of Table 1 (as indicated in FIG. 2) will provide the basic alignment of the candidate protein against the CMT3 catalytic region. Candidate CMT3 proteins with at least 50% identity at the conserved amino acid positions, preferably at least 50% to 60%, 60% to 70%, 70% to 80%, 80% to 90%, 90% to 100%, or 100% are identified as CMT3 group proteins by this method. Candidate proteins with these amounts of identity at these conserved positions and which have DNA methyltransferase activity in vitro or in vivo are useful for certain embodiments of the present invention.

Having illustrated and described the principles of the present invention, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims. Within a species, either independent transformations of varieties within the species or crossing/backcrossing from a transformed variety into other varieties provides for introducing the transgenes into all the varieties within a species. The use of inducible promoters to expression recombinant DNA methyltransferases is considered a non-limiting embodiment of the present invention.

TABLE 1
DRM2 and CMET3 Methyltransferases in plants.

| | Protein | |
|---|---|---|
| Plant Species | DRM2 | CMT3 |
| Arabidopsis thaliana | NP 196966.2 | NP 177135.1 |
| Capsella rubella | XP 006287272.1 | XP 006300392.1 |
| Ricinus communis | XP 002521449.1 | XP 002530367.1 |
| Solanum tuberosum | XP 006346949.1 | XP 006354167.1 |
| Citrus clementina | XP 006446539.1 | XP 006445885.1 |
| Citrus sinensis | AGU16983.1 | NP 001275877.1 |
| Solanum lycopersicum | XP 004237065.1 | XP 004252840.1 |
| Vitis vinifera | XP 002273972.2 | XP 002283355.2 |
| Fragaria vesca subsp. Vesca | XP 004304636.1 | XP 004288717.1 |
| Phaseolus vulgaris | XP 007151016.1 | XP 007152975.1 |
| Populus trichocarpa | XP 002300046.2 | XP 002299134.2 |
| Glycine max | XP 003524549.1 | XP 006572936.1 |
| Hordeum vulgare subsp. Vulgare | BAJ96312.1 | CAJ01708.1 |
| Oryza sativa | ABF93591.1 | EEE58631.1 |
| Sorghum bicolor | XP 002468660.1 | XP 002448525.1 |
| Zea mays | NP 001104977.1 | NP 001104978.1 |
| Triticum urartu | EMS60441.1 | |
| Aegilops tauschii | EMT00800.1 | |

What is claimed is:

1) A method for producing a crop plant exhibiting a useful trait in comparison to a control plant comprising the steps of:
(a) expressing in a first plant or plant cell a recombinant DNA methyltransferase that is a member of the group consisting of DRM and CMT3 DNA methyltransferases and their catalytic domains;
(b) selfing or crossing a first plant of step (a) or a plant derived from a plant cell of step (a), or progeny thereof, to a second plant to produce progeny; and,
(c) selecting progeny of step (b), or progeny thereof, that exhibit a useful trait and lack a recombinant DNA methyltransferase.

2) The method of claim 1, wherein the second plant of step (b) is isogenic to the plant or plant cell of step (a).

3) The method of claim 1, wherein progeny of step (c) are hybrids and have increased yields relative to a control hybrid plant.

4) The method of claim 1, wherein the progeny, or progeny thereof, of step (c) are vegetatively propagated.

5) The method of claim 1, wherein the parent plant of step (a) and/or at least a portion of the progeny plants of step (b) or step (c) exhibit one or more MSH1-dr phenotypes.

6) The method of claim 1, wherein said recombinant DNA methyltransferase is a member of the group consisting of DRM and CMT3 DNA methyltransferases and catalytic domains of DRM and CMT3 DNA methyltransferases comprising at least 90% sequence identity to the conserved amino acids identified in FIG. 1 or FIG. 2.

7) The method of claim 1, wherein the plant is a crop plant selected from the group consisting of corn, soybean, cotton, wheat, rice, tomato, tobacco, millet, potato, sorghum, alfalfa, sunflower, canola, peanut, canola (Brassica napus, Brassica rapa ssp.), coffee (Coffea spp.), coconut (Cocos nucijra), pineapple (Ananas comosus), citrus trees (Citrus spp.), cocoa (Theobroma cacao), poplar, sugar beets (Beta vulgaris), sugarcane (Saccharum spp.), oats, barley, vegetables, ornamentals, and conifers.

8) A crop plant comprising a recombinant DNA methyltransferase, wherein said recombinant DNA methyltransferase is a member of the group consisting of DRM and CMT3 DNA methyltransferases and catalytic domains of DRM and CMT3 DNA methyltransferases.

9) A crop plant of claim 8 comprising a recombinant DNA methyltransferase, wherein said recombinant DNA methyltransferase is a member of the group consisting of DRM and CMT3 DNA methyltransferases and catalytic domains of DRM and CMT3 DNA methyltransferases comprising at least 90% sequence identity to the conserved amino acids identified in FIG. 1 or FIG. 2.

10) A crop plant of claim 8 comprising a recombinant DNA methyltransferase, wherein said recombinant DNA methyltransferase is a member of the group consisting of DRM and CMT3 DNA methyltransferases and catalytic domains of DRM and CMT3 DNA methyltransferases provided in Table 1.

11) A recombinant DNA methyltransferase comprising a plant promoter operably linked to a transcribed region comprising a DNA methyltransferase that is a member of the group consisting of DRM and CMT3 DNA methyltransferases and catalytic domains of DRM and CMT3 DNA methyltransferases comprising at least 90%, at least 95%, at least 98%, or 100% sequence identity to the conserved amino acids identified in FIG. 1 or FIG. 2.

12) A recombinant DNA construct of claim 11 comprising a promoter that is selectively expressed in cells containing sensory plastids.

13) The recombinant DNA construct of claim 12, wherein the promoter is selected from the group consisting of Msh1, PPD3, or PSBO1, or PSBO2 promoters.


SEQUENCE LISTING (31 sequences)

SEQ ID NO: 1 - DNA, 589 bp, Streptomyces hygroscopicus
  5′UTR (1)..(24)
  CDS (25)..(576)
  3′UTR (577)..(589)
atatctcgagcccggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgaatcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccacgcggcatgctgcgggcggccggcttcaagcacgggaactggcatgacgtgggtttctggcagctggacttcagcctgccagtaccgccccgtccggtcctgcccgtcaccgaaatctgatgactcgagatat

SEQ ID NO: 2 - protein, 183 aa, Streptomyces hygroscopicus
MSPERRPADIRRATEADMPAVCTIVNHYIETSTVNFRTEPQEPQEWTDDLVRLRERYPWLVAEVDGEVAGIAYAGPWKARNAYDWTAESTVYVSPRHQRTGLGSTLYTHLLKSLEAQGFKSVVAVIGLPNDPSVRMHEALGYAPRGMLRAAGFKHGNWHDVGFWQLDFSLPVPPRPVLPVTEI

SEQ ID NO: 3 - DNA, 441 bp, Cauliflower mosaic virus
  promoter (1)..(441): flanking restrictions sites
atatggatccatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctctcgagatat

SEQ ID NO: 4 - DNA, 818 bp, Artificial Sequence
  synthetic GFP with N-terminal duplicated nuclear localization signal and no stop codon
atatctcgagcacaccaccatgggcgatccaaagaagaagagaaaggtagaccctaagaagaagcgtaaagtcccaggcatggtgagcaagggcgaggagctgttcactggggtggtgcccatcctggtcgagctggacggcgacgtgaacggccacaagttcagcgtccgaggcgagggcgagggcgacgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggcacctacaagacccgagccgaggtgaagttcgagggcgacaccctggtgaaccgcatcgagctgaagggcatcgacttcaaggaggacggcaacatcctggggcacaagctggagtacaacttcaacagccacaacgtctacatcacggctgacaagcagaagaacggcatcaaggccaacttcaagatccgccacaacgtcgaggacggcagcgtccagctcgccgaccactaccagcagaacacgcccatcggcgacggtcccgtgctgctgcccgacaaccactacctgagccaccagtccgctctgagcaaggaccccaacgagaagcgcgaccacatggtcctgctggagttcgtcaccgcagctggcatcacccacggcatggacgagctgtacaagccgcggaaatttggtaccatat

SEQ ID NO: 5 - protein, 20 aa, Artificial Sequence
  Synthetic Construct
MGDPKKKRKVDPKKKRKVPG

SEQ ID NO: 6 - protein, 239 aa, Artificial Sequence
  Synthetic Construct
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAAGITHGMDELYK

SEQ ID NO: 7 - DNA, 2109 bp, Artificial Sequence
  Soybean DRM2 CDS with NOS 3′ PolyA region
atatccgcggatgggaggagatgattctggtttggagagtgacaattttgattggaacactgaagatgagcttgaaattcagaactataactcgtcgtcttcatgtttaacccttcctaatggagatgctgttactggctctggagaggcaagctcgtctgcagttttggctaattccaaggtgcttgatcacttcgtcagcatgggattttctagagaaatggtttctaaagtaattcaggaatatggtgaggaaaatgaagataaactacttgaagaacttctcacatacaaggctctagaaagttcttcccgtccacagcagcgaattgagccagatccttgttcttcagagaatgcagggagttcttgggatgatttctcagatactgatattttttctgatgatgaagaaattgcaaaaactatgtctgagaatgatgataccttacggtctttggtgaaaatggggtacaagcaggtggaggctttaattgccatagaaagattaggcccaaacgcctcacttgaagaattggtagattttataggtgttgctcaaatggcaaaggctgaagatgctcttctgcctcctcaagaaaagttacaatacaatgactatgctaagtccaataaacgaagattatatgattatgaagtgctgggaaggaaaaagcctaggggatgtgagaagaaaatcctcaatgaagatgacgaggatgctgaagctcttcatcttccaaatccgatgattgggtttggtgtacctacagagtcaagcttcataacacacagaaggcttccagaagatgccattgggcctccttacttctattacgagaatgtagccttagcaccaaaaggtgtttggcaaacaatttcaagattcttgtatgatgttgaacctgagtttgtagattcgaagtttttctgtgctgcagccaggaaaaggggatatattcacaatctccccatccaaaataggttcccacttctaccacttccaccgcgcacaatacatgaggcttttcccctaacaaagaaatggtggccctcatgggatattaggaccaagcttaattgtttgcaaacatgtattggcagtgcaaaactaacagaaagaattaggaaagctgtagaaatctatgatgaagatccacctgaaagtgtacagaagtatgttcttcatcagtgtaggaaatggaatttggtttgggtgggaagaaataaggttgctccattagagcctgatgaagtagaaacgctgttgggcttcccaaggaaccacaccagaggaggtgggataagtaggactgacagatacaagtcacttggtaattcattccaggttgacactgtggcataccacttgtcagttctgaaggagatgtatcctaatggtatcaatcttctttctctcttttctggaattggtggggcggaggtagctcttcatcggcttggcatccctctcaagaatgttgtgtctgttgaaaaatcggaagttaataggaatattgttagaagttggtgggagcaaacaaaccagaaaggtaatctatatgatatggatgatgtaagggagctagacggtgatcgcttggaacagctgatgagcacatttggtggttttgatctaattgttggtggcagtccatgtaataacctggctggtagcaacagggtcagccgggatggactggagggaaaagaatcttctctcttctttgattattttaggattctagacttggtaaaaaatatgtcggccaaatatcgatgaaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 8 - protein, 590 aa, Artificial Sequence
  Synthetic Construct
MGGDDSGLESDNFDWNTEDELEIQNYNSSSSCLTLPNGDAVTGSGEASSSAVLANSKVLDHFVSMGFSREMVSKVIQEYGEENEDKLLEELLTYKALESSSRPQQRIEPDPCSSENAGSSWDDFSDTDIFSDDEEIAKTMSENDDTLRSLVKMGYKQVEALIAIERLGPNASLEELVDFIGVAQMAKAEDALLPPQEKLQYNDYAKSNKRRLYDYEVLGRKKPRGCEKKILNEDDEDAEALHLPNPMIGFGVPTESSFITHRRLPEDAIGPPYFYYENVALAPKGVWQTISRFLYDVEPEFVDSKFFCAAARKRGYIHNLPIQNRFPLLPLPPRTIHEAFPLTKKWWPSWDIRTKLNCLQTCIGSAKLTERIRKAVEIYDEDPPESVQKYVLHQCRKWNLVWVGRNKVAPLEPDEVETLLGFPRNHTRGGGISRTDRYKSLGNSFQVDTVAYHLSVLKEMYPNGINLLSLFSGIGGAEVALHRLGIPLKNVVSVEKSEVNRNIVRSWWEQTNQKGNLYDMDDVRELDGDRLEQLMSTFGGFDLIVGGSPCNNLAGSNRVSRDGLEGKESSLFFDYFRILDLVKNMSAKYR

SEQ ID NO: 9 - DNA, 1488 bp, Artificial Sequence
  Soybean DRM2 catalytic region attached to NOS3′ polyA region
atatccgcggaataaacgaagattatatgattatgaagtgctgggaaggaaaaagcctaggggatgtgagaagaaaatcctcaatgaagatgacgaggatgctgaagctcttcatcttccaaatccgatgattgggtttggtgtacctacagagtcaagcttcataacacacagaaggcttccagaagatgccattgggcctccttacttctattacgagaatgtagccttagcaccaaaaggtgtttggcaaacaatttcaagattcttgtatgatgttgaacctgagtttgtagattcgaagtttttctgtgctgcagccaggaaaaggggatatattcacaatctccccatccaaaataggttcccacttctaccacttccaccgcgcacaatacatgaggcttttcccctaacaaagaaatggtggccctcatgggatattaggaccaagcttaattgtttgcaaacatgtattggcagtgcaaaactaacagaaagaattaggaaagctgtagaaatctatgatgaagatccacctgaaagtgtacagaagtatgttcttcatcagtgtaggaaatggaatttggtttgggtgggaagaaataaggttgctccattagagcctgatgaagtagaaacgctgttgggcttcccaaggaaccacaccagaggaggtgggataagtaggactgacagatacaagtcacttggtaattcattccaggttgacactgtggcataccacttgtcagttctgaaggagatgtatcctaatggtatcaatcttctttctctcttttctggaattggtggggcggaggtagctcttcatcggcttggcatccctctcaagaatgttgtgtctgttgaaaaatcggaagttaataggaatattgttagaagttggtgggagcaaacaaaccagaaaggtaatctatatgatatggatgatgtaagggagctagacggtgatcgcttggaacagctgatgagcacatttggtggttttgatctaattgttggtggcagtccatgtaataacctggctggtagcaacagggtcagccgggatggactggagggaaaagaatcttctctcttctttgattattttaggattctagacttggtaaaaaatatgtcggccaaatatcgatgaaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 10 - protein, 383 aa, Artificial Sequence
  Synthetic Construct
NKRRLYDYEVLGRKKPRGCEKKILNEDDEDAEALHLPNPMIGFGVPTESSFITHRRLPEDAIGPPYFYYENVALAPKGVWQTISRFLYDVEPEFVDSKFFCAAARKRGYIHNLPIQNRFPLLPLPPRTIHEAFPLTKKWWPSWDIRTKLNCLQTCIGSAKLTERIRKAVEIYDEDPPESVQKYVLHQCRKWNLVWVGRNKVAPLEPDEVETLLGFPRNHTRGGGISRTDRYKSLGNSFQVDTVAYHLSVLKEMYPNGINLLSLFSGIGGAEVALHRLGIPLKNVVSVEKSEVNRNIVRSWWEQTNQKGNLYDMDDVRELDGDRLEQLMSTFGGFDLIVGGSPCNNLAGSNRVSRDGLEGKESSLFFDYFRILDLVKNMSAKYR

SEQ ID NO: 11 - DNA, 2964 bp, Artificial Sequence
  Soybean CMT3 coding region and flanking NOS3′ polyA region
atatccgcggatgcctagcaagcgcaagaccagatcctccgcctctcccgctgccgcgccgccgagcaagcgcgcctccagatcctccgcctctcgcgtcgccgattccgcaccggtcaaatctgaagccgaggaagttgtggcagcttcctctgtcgtcaaagaagaggcgcaagcaagcttcacggacgttactgacggcaacgttagcgatggcgaggggactaacgccagattcgtcggagagcctgttcccgacgaggaagcccggcgccgctggccgaaacggtaccaggaaaaggaaaagaagcaatccgctgggccaaaatcaaacagaaacgatgaagacgaggagattcaacaagctcgacgtcactacactcaggcagaagtagatgggtgcatgctttacaaactttacgatgatgcccatgttaaagcagaagaaggagaagacaattacatttgtaaaattgttgagatatttgaggccattgatggggcactgtattttacggcacaatggtattatagggctaaagacactgtcattaaaaaacttgcatatctcattgaaccaaagcgagttttcttttctgaagtccaggatgacaaccctttggattgtctagttgaaaagctgaacatcgccagaataacattaaatgtagatttagaagcaaagaaggaaaccattccaccttgtgattattactgtgatacacaatatcttttgccatactccacatttgttaacttaccatcagaaaatggggaatctggtagtgaaacttcttctacaatatcttctgaaactaatggaatcggaaaatatgaggtgaactctcaacctaaggaagcttttcttcccgaagaaagtaaagatccggagatgaagttactagatttatattgtggttgtggtgcaatgtcaactggtttgtgccttggtggaaatttatctggtgtgaaccttgttactagatgggcagtggacttgaatcaacatgcttgtgaatgtcttaaattaaaccatcctgaaactgaggttagaaatgaatcggcagaaaattttctttcattattgaaggagtggcaggaattatgtagttacttctctctagttgaaaaaaaggtgtcacatgagaaatatgtgaatctttttagtgaagatgacgatgacactagcagtaatgaagaggttaatagtgaagatgacaatgaactgaatgaagatgatgaaatatttgaagtttctgaaatccttgctgtctgctacggtgacccaaataagaaaaaagaacaagggttatacttcaaggttcattggaagggttatgaatctgccctggattcttgggaaccaattgaaggtctaagtaattgcaaggaaaagattaaagaatttgtcagtcgaggcttcaagtcacagatattgcctttgcctggagatgttgatgtaatttgtggtggacctccttgccaaggtattagtggtttcaaccggtttcggaacaaagagagtcctttggatgatgagaagaacaaacaactagttgtttttatggatattgttcaataccttaagcccaaatttacattgatggaaaatgtggttgatcttgtaaaatttgcggaaggctttcttgggagatatgccttgggtcgccttcttcaaatgaattatcaagcgcgtttaggaattatggctgcaggtgcttatgggcttcctcagtttcgtttgcgcgtctttttatggggggctgcaccttctcagaagttgccacaatttccgcttccaactcatgatgttattgtaaggggtgttattcccttggagtttgagataaacactgtagcatacaatgaaggacaaaaggttcaactgcagaagaagcttttattggaggatgctatttctgaccttcctcgggttcagaacaatgagcgtcgtgatgagataaaatatgacaaagctgctcaaacggagttccaacgattcattagattaagcaaacatgaaatgttggagcttcaatctagaacaaaatcgtcgaagtctttgctatatgatcatcgtccactagaattgaatgcggatgattaccaacgtgtgtgtcggatccctaaaaagaagggtggatgcttcagagatttaccaggtgttcgtgtgggagctgataacaaggttgaatgggatcctgatgtcgaacgtgtatatttggattcaggaaaaccattggttccagattatgccatgacttttgtgaatggaacttcatcaaaaccttttgctcggttatggtgggatgaaactgttccaactgttgtgacaagagcagaacctcacaaccaggcaattttacaccctgaacaagacagagtgttgacgattcgtgaaaatgcaagactccaaggttttccagatttctacaagttgtgtgggccggtcaaagaaaggtacattcaagttgggaatgcagtggcagttccagtagctagagctttaggatacacactaggccttgcatttgaagggtctacttctacaagtgatgatccattgtataaattacctgataaatttcccatgattagggatcgggtttcttctgtatcttccgaagatgatgtgtaaaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 12 - protein, 875 aa, Artificial Sequence
  Synthetic Construct
MPSKRKTRSSASPAAAPPSKRASRSSASRVADSAPVKSEAEEVVAASSVVKEEAQASFTDVTDGNVSDGEGTNARFVGEPVPDEEARRRWPKRYQEKEKKQSAGPKSNRNDEDEEIQQARRHYTQAEVDGCMLYKLYDDAHVKAEEGEDNYICKIVEIFEAIDGALYFTAQWYYRAKDTVIKKLAYLIEPKRVFFSEVQDDNPLDCLVEKLNIARITLNVDLEAKKETIPPCDYYCDTQYLLPYSTFVNLPSENGESGSETSSTISSETNGIGKYEVNSQPKEAFLPEESKDPEMKLLDLYCGCGAMSTGLCLGGNLSGVNLVTRWAVDLNQHACECLKLNHPETEVRNESAENFLSLLKEWQELCSYFSLVEKKVSHEKYVNLFSEDDDDTSSNEEVNSEDDNELNEDDEIFEVSEILAVCYGDPNKKKEQGLYFKVHWKGYESALDSWEPIEGLSNCKEKIKEFVSRGFKSQILPLPGDVDVICGGPPCQGISGFNRFRNKESPLDDEKNKQLVVFMDIVQYLKPKFTLMENVVDLVKFAEGFLGRYALGRLLQMNYQARLGIMAAGAYGLPQFRLRVFLWGAAPSQKLPQFPLPTHDVIVRGVIPLEFEINTVAYNEGQKVQLQKKLLLEDAISDLPRVQNNERRDEIKYDKAAQTEFQRFIRLSKHEMLELQSRTKSSKSLLYDHRPLELNADDYQRVCRIPKKKGGCFRDLPGVRVGADNKVEWDPDVERVYLDSGKPLVPDYAMTFVNGTSSKPFARLWWDETVPTVVTRAEPHNQAILHPEQDRVLTIRENARLQGFPDFYKLCGPVKERYIQVGNAVAVPVARALGYTLGLAFEGSTSTSDDPLYKLPDKFPMIRDRVSSVSSEDDV

SEQ ID NO: 13 - DNA, 2085 bp, Artificial Sequence
  Soybean catalytic CMT3 domain and NOS 3′ polyA region
atatccgcgggagatgaagttactagatttatattgtggttgtggtgcaatgtcaactggtttgtgccttggtggaaatttatctggtgtgaaccttgttactagatgggcagtggacttgaatcaacatgcttgtgaatgtcttaaattaaaccatcctgaaactgaggttagaaatgaatcggcagaaaattttctttcattattgaaggagtggcaggaattatgtagttacttctctctagttgaaaaaaaggtgtcacatgagaaatatgtgaatctttttagtgaagatgacgatgacactagcagtaatgaagaggttaatagtgaagatgacaatgaactgaatgaagatgatgaaatatttgaagtttctgaaatccttgctgtctgctacggtgacccaaataagaaaaaagaacaagggttatacttcaaggttcattggaagggttatgaatctgccctggattcttgggaaccaattgaaggtctaagtaattgcaaggaaaagattaaagaatttgtcagtcgaggcttcaagtcacagatattgcctttgcctggagatgttgatgtaatttgtggtggacctccttgccaaggtattagtggtttcaaccggtttcggaacaaagagagtcctttggatgatgagaagaacaaacaactagttgtttttatggatattgttcaataccttaagcccaaatttacattgatggaaaatgtggttgatcttgtaaaatttgcggaaggctttcttgggagatatgccttgggtcgccttcttcaaatgaattatcaagcgcgtttaggaattatggctgcaggtgcttatgggcttcctcagtttcgtttgcgcgtctttttatggggggctgcaccttctcagaagttgccacaatttccgcttccaactcatgatgttattgtaaggggtgttattcccttggagtttgagataaacactgtagcatacaatgaaggacaaaaggttcaactgcagaagaagcttttattggaggatgctatttctgaccttcctcgggttcagaacaatgagcgtcgtgatgagataaaatatgacaaagctgctcaaacggagttccaacgattcattagattaagcaaacatgaaatgttggagcttcaatctagaacaaaatcgtcgaagtctttgctatatgatcatcgtccactagaattgaatgcggatgattaccaacgtgtgtgtcggatccctaaaaagaagggtggatgcttcagagatttaccaggtgttcgtgtgggagctgataacaaggttgaatgggatcctgatgtcgaacgtgtatatttggattcaggaaaaccattggttccagattatgccatgacttttgtgaatggaacttcatcaaaaccttttgctcggttatggtgggatgaaactgttccaactgttgtgacaagagcagaacctcacaaccaggcaattttacaccctgaacaagacagagtgttgacgattcgtgaaaatgcaagactccaaggttttccagatttctacaagttgtgtgggccggtcaaagaaaggtacattcaagttgggaatgcagtggcagttccagtagctagagctttaggatacacactaggccttgcatttgaagggtctacttctacaagtgatgatccattgtataaattacctgataaatttcccatgattagggatcgggtttcttctgtatcttccgaagatgatgtgtaaaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 14 - protein, 582 aa, Artificial Sequence
  Synthetic Construct
EMKLLDLYCGCGAMSTGLCLGGNLSGVNLVTRWAVDLNQHACECLKLNHPETEVRNESAENFLSLLKEWQELCSYFSLVEKKVSHEKYVNLFSEDDDDTSSNEEVNSEDDNELNEDDEIFEVSEILAVCYGDPNKKKEQGLYFKVHWKGYESALDSWEPIEGLSNCKEKIKEFVSRGFKSQILPLPGDVDVICGGPPCQGISGFNRFRNKESPLDDEKNKQLVVFMDIVQYLKPKFTLMENVVDLVKFAEGFLGRYALGRLLQMNYQARLGIMAAGAYGLPQFRLRVFLWGAAPSQKLPQFPLPTHDVIVRGVIPLEFEINTVAYNEGQKVQLQKKLLLEDAISDLPRVQNNERRDEIKYDKAAQTEFQRFIRLSKHEMLELQSRTKSSKSLLYDHRPLELNADDYQRVCRIPKKKGGCFRDLPGVRVGADNKVEWDPDVERVYLDSGKPLVPDYAMTFVNGTSSKPFARLWWDETVPTVVTRAEPHNQAILHPEQDRVLTIRENARLQGFPDFYKLCGPVKERYIQVGNAVAVPVARALGYTLGLAFEGSTSTSDDPLYKLPDKFPMIRDRVSSVSSEDDV

SEQ ID NO: 15 - DNA, 1579 bp, Zea mays
  misc_feature (1)..(10): BamHI site
atatggatccgcatgcaagctgatccactagaggccatggcggccgcactaggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtactagttgtcgacatat

SEQ ID NO: 16 - DNA, 2148 bp, Artificial Sequence
  Maize DRM2 coding sequence and NOS 3′ polyA region
atatccgcggatggtgcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggttgctaatggcaatgggaaggctgggcgatctgcctctttggttcagaagtatgtggacatgggtttctcagaagagattgttctgaaggccatgaaggacaatggggataatggagcagattcattagttgagctccttcttacttaccaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggatttcttacatgagatgtcacggaaggacgaaaaagttgattccttagttaaaatggggtttcctgaagacgaggctgcactggctattaccagatgcgggccggatgcatctatttctgttctggtggattcaatctatgcttcacagaccgcaggagatggttactgtggcaatctgtctgactatgaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccaccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacggatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 17 - protein, 603 aa, Artificial Sequence
  Synthetic Construct
MVHWVSDSDGSDNFEWDSDGNGEQTVSFNAAGAGSSALAATNTDAPGPSTRVANGNGKAGRSASLVQKYVDMGFSEEIVLKAMKDNGDNGADSLVELLLTYQELGNDLKVDNDFASSCAPKTADDSDDDDTLEIWDDEDAGGRSTRVANSVDDSDDEDFLHEMSRKDEKVDSLVKMGFPEDEAALAITRCGPDASISVLVDSIYASQTAGDGYCGNLSDYEDNSYGGRSTGNKKKRKRYGGQAQGSRGPLDGSCDEPMPLPHPMVGFNLPDQWSRRVDRSLPAQAIGPPYFYYENVALAPKGVWTTISRFLYDIQPEFVDSKYFCAAARKRGYIHNLPLENRSPLLPIPPKTISEAFPRTKRWWPSWDPRRQFNCLQTCVSSAKLLERIRVALTNSSDPPPPRVQKYVLEECRKWNLAWVGLNKVAPLEPDEMEFLLGFPKDHTRGISRTERYRSLGNSFQVDTVAYHLSVLKDLFPQGMNVLSLFSGIGGAEVALHRLGIRMNTVISVEKSEVNRTILKSWWDQTQTGTLIEITDVQTLSSERIEAYIRRIGGFDLVIGGSPCNNLTGSNRHHRDGLEGEHSALFHHYFRILHAVKSIMERL

SEQ ID NO: 18 - DNA, 1404 bp, Artificial Sequence
  Maize DRM2 catalytic CDS attached to NOS 3′ polyA region
atatccgcggccattagatggtagctgtgatgaacccatgcctctcccacatccaatggttggatttaacttgccagaccagtggtcaagacgagtggacagatcgttgcctgcacaagctattggtccaccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacggatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 19 - protein, 355 aa, Artificial Sequence
  Synthetic Construct
PLDGSCDEPMPLPHPMVGFNLPDQWSRRVDRSLPAQAIGPPYFYYENVALAPKGVWTTISRFLYDIQPEFVDSKYFCAAARKRGYIHNLPLENRSPLLPIPPKTISEAFPRTKRWWPSWDPRRQFNCLQTCVSSAKLLERIRVALTNSSDPPPPRVQKYVLEECRKWNLAWVGLNKVAPLEPDEMEFLLGFPKDHTRGISRTERYRSLGNSFQVDTVAYHLSVLKDLFPQGMNVLSLFSGIGGAEVALHRLGIRMNTVISVEKSEVNRTILKSWWDQTQTGTLIEITDVQTLSSERIEAYIRRIGGFDLVIGGSPCNNLTGSNRHHRDGLEGEHSALFHHYFRILHAVKSIMERL

SEQ ID NO: 20 - DNA, 3075 bp, Artificial Sequence
  Maize CMT3 coding region attached to NOS 3′ polyA site
atatccgcggatggcgccgagctccccgtcacccgccgcgcctacacgcgtctctgggcggaagcgcgccgccaaggccgaggagatccaccagaacaaggaggaggaggaggaggtcgcggcggcgtcctccgccaagcgcagccgcaaggcggcatcttccgggaagaagcccaagtcgccccccaagcaggccaagccggggaggaagaagaagggggatgccgagatgaaggagcccgtggaggacgacgtgtgcgccgaggagcccgacgaggaggagttggccatgggcgaggaggaggccgaggagcaggccatgcaggaggaggtggttgcggtcgcggcggggtcacccgggaagaagagggtggggagaaggaacgccgccgccgccgctggcgaccacgagccggagttcatcggcagccctgttgccgccgacgaggcgcgcagcaactggcccaagcgctacggccgcagcactgccgcaaagaaaccggatgaggaggaagagctcaaggccagatgtcactaccggagcgctaaggtggacaacgtcgtctactgcctcggggatgacgtctatgtcaaggctggagaaaacgaggcagattacattggccgcattactgaattttttgaggggactgaccagtgtcactattttacttgccgttggttcttccgagcagaggacacggttatcaattctttggtgtccataagtgtggatggccacaagcatgaccctagacgtgtttttctttctgaggaaaagaacgacaatgtgcttgattgcattatctccaaggtcaagatagtccatgttgatccaaatatggatccaaaagccaaggctcagctgatagagagttgcgacctatactatgacatgtcttactctgttgcatattctacatttgctaatatctcgtctgaaaatgggcagtcaggcagtgataccgcttcgggtatttcttctgatgatgtggatctggagacgtcatctagtatgccaacgaggacagcaacccttcttgatctgtattctggctgtgggggcatgtctactggtctttgcttgggtgcagctctttctggcttgaaacttgaaactcgatgggctgttgatttcaacagttttgcgtgccaaagtttaaaatataatcatccacagactgaggtgcgaaatgagaaagccgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtccagtgggaaggatacggccctgaggaggatacatgggaaccgattgataacttgagtgactgcccgcagaaaattagagaatttgtacaagaagggcacaaaagaaagattctcccactgcctggtgatgttgatgtcatttgtggaggcccaccatgccaaggtatcagtgggtttaatcggtacagaaaccgtgatgagccactcaaagatgagaaaaacaaacaaatggtgactttcatggatattgtggcgtacttgaagcccaagtatgttctcatggaaaatgtggtggacatactcaaatttgcggatggttacctaggaaaatatgctttgagctgccttgttgctatgaagtaccaagcgcggcttggaatgatggtggctggttgctatggtctgccacagttcaggatgcgtgtgttcctctggggtgctctttcttccatggtgctccctaagtatcctctgcccacctatgatgttgtagtacgtggaggagcccctaatgccttttcgcaatgtatggttgcatatgacgagacacaaaaaccatccctgaaaaaagccttgcttcttggcgatgcaatttcagatttaccaaaggttcaaaatcaccagcctaacgatgtgatggagtatggtggttcccccaagaccgaattccagcgctacattcgactcagtcgtaaagacatgttggattggtccttcggtgagggggctggtccagatgaaggcaagctcttggatcaccagcctttacggcttaacaacgatgattatgagcgggttcaacagattcctgtcaagaagggagccaacttccgcgacctaaagggcgtgagggttggagcaaacaatattgttgagtgggatccagaaatcgagcgtgtgaaactttcatctgggaaaccactggttcctgactatgcaatgtcattcatcaagggcaaatcactcaagccgtttgggcgcctgtggtgggacgagacagttcctacagttgtaaccagagcagagcctcacaaccaggttataattcatccgactcaagcaagggtcctcactatccgggagaacgcaaggttacagggcttccccgattactaccgattgtttggcccgatcaaggagaagtacattcaagtcgggaacgcagtggctgtccctgttgcccgggcactgggctactgtctggggcaagcctacctgggtgaatctgaggggagtgaccctctgtaccagctgcctccaagtttcacctctgttggaggacgcactgcggggcaggcgagggcctctcctgttggcaccccagcaggggaggtagttgagcagtaaaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 21 - protein, 912 aa, Artificial Sequence
  Synthetic Construct
MAPSSPSPAAPTRVSGRKRAAKAEEIHQNKEEEEEVAAASSAKRSRKAASSGKKPKSPPKQAKPGRKKKGDAEMKEPVEDDVCAEEPDEEELAMGEEEAEEQAMQEEVVAVAAGSPGKKRVGRRNAAAAAGDHEPEFIGSPVAADEARSNWPKRYGRSTAAKKPDEEEELKARCHYRSAKVDNVVYCLGDDVYVKAGENEADYIGRITEFFEGTDQCHYFTCRWFFRAEDTVINSLVSISVDGHKHDPRRVFLSEEKNDNVLDCIISKVKIVHVDPNMDPKAKAQLIESCDLYYDMSYSVAYSTFANISSENGQSGSDTASGISSDDVDLETSSSMPTRTATLLDLYSGCGGMSTGLCLGAALSGLKLETRWAVDFNSFACQSLKYNHPQTEVRNEKADEFLALLKEWAVLCKKYVQDVDSNLASSEDQADEDSPLDKDEFVVEKLVGICYGGSDRENGIYFKVQWEGYGPEEDTWEPIDNLSDCPQKIREFVQEGHKRKILPLPGDVDVICGGPPCQGISGFNRYRNRDEPLKDEKNKQMVTFMDIVAYLKPKYVLMENVVDILKFADGYLGKYALSCLVAMKYQARLGMMVAGCYGLPQFRMRVFLWGALSSMVLPKYPLPTYDVVVRGGAPNAFSQCMVAYDETQKPSLKKALLLGDAISDLPKVQNHQPNDVMEYGGSPKTEFQRYIRLSRKDMLDWSFGEGAGPDEGKLLDHQPLRLNNDDYERVQQIPVKKGANFRDLKGVRVGANNIVEWDPEIERVKLSSGKPLVPDYAMSFIKGKSLKPFGRLWWDETVPTVVTRAEPHNQVIIHPTQARVLTIRENARLQGFPDYYRLFGPIKEKYIQVGNAVAVPVARALGYCLGQAYLGESEGSDPLYQLPPSFTSVGGRTAGQARASPVGTPAGEVVEQ

SEQ ID NO: 22 - DNA, 2055 bp, Artificial Sequence
  catalytic domain of Maize CMT3 attached to NOS 3′ polyA region
atatccgcgggcaacccttcttgatctgtattctggctgtgggggcatgtctactggtctttgcttgggtgcagctctttctggcttgaaacttgaaactcgatgggctgttgatttcaacagttttgcgtgccaaagtttaaaatataatcatccacagactgaggtgcgaaatgagaaagccgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtccagtgggaaggatacggccctgaggaggatacatgggaaccgattgataacttgagtgactgcccgcagaaaattagagaatttgtacaagaagggcacaaaagaaagattctcccactgcctggtgatgttgatgtcatttgtggaggcccaccatgccaaggtatcagtgggtttaatcggtacagaaaccgtgatgagccactcaaagatgagaaaaacaaacaaatggtgactttcatggatattgtggcgtacttgaagcccaagtatgttctcatggaaaatgtggtggacatactcaaatttgcggatggttacctaggaaaatatgctttgagctgccttgttgctatgaagtaccaagcgcggcttggaatgatggtggctggttgctatggtctgccacagttcaggatgcgtgtgttcctctggggtgctctttcttccatggtgctccctaagtatcctctgcccacctatgatgttgtagtacgtggaggagcccctaatgccttttcgcaatgtatggttgcatatgacgagacacaaaaaccatccctgaaaaaagccttgcttcttggcgatgcaatttcagatttaccaaaggttcaaaatcaccagcctaacgatgtgatggagtatggtggttcccccaagaccgaattccagcgctacattcgactcagtcgtaaagacatgttggattggtccttcggtgagggggctggtccagatgaaggcaagctcttggatcaccagcctttacggcttaacaacgatgattatgagcgggttcaacagattcctgtcaagaagggagccaacttccgcgacctaaagggcgtgagggttggagcaaacaatattgttgagtgggatccagaaatcgagcgtgtgaaactttcatctgggaaaccactggttcctgactatgcaatgtcattcatcaagggcaaatcactcaagccgtttgggcgcctgtggtgggacgagacagttcctacagttgtaaccagagcagagcctcacaaccaggttataattcatccgactcaagcaagggtcctcactatccgggagaacgcaaggttacagggcttccccgattactaccgattgtttggcccgatcaaggagaagtacattcaagtcgggaacgcagtggctgtccctgttgcccgggcactgggctactgtctggggcaagcctacctgggtgaatctgaggggagtgaccctctgtaccagctgcctccaagtttcacctctgttggaggacgcactgcggggcaggcgagggcctctcctgttggcaccccagcaggggaggtagttgagcagtaaaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggatat

SEQ ID NO: 23 - protein, 572 aa, Artificial Sequence
  Synthetic Construct
ATLLDLYSGCGGMSTGLCLGAALSGLKLETRWAVDFNSFACQSLKYNHPQTEVRNEKADEFLALLKEWAVLCKKYVQDVDSNLASSEDQADEDSPLDKDEFVVEKLVGICYGGSDRENGIYFKVQWEGYGPEEDTWEPIDNLSDCPQKIREFVQEGHKRKILPLPGDVDVICGGPPCQGISGFNRYRNRDEPLKDEKNKQMVTFMDIVAYLKPKYVLMENVVDILKFADGYLGKYALSCLVAMKYQARLGMMVAGCYGLPQFRMRVFLWGALSSMVLPKYPLPTYDVVVRGGAPNAFSQCMVAYDETQKPSLKKALLLGDAISDLPKVQNHQPNDVMEYGGSPKTEFQRYIRLSRKDMLDWSFGEGAGPDEGKLLDHQPLRLNNDDYERVQQIPVKKGANFRDLKGVRVGANNIVEWDPEIERVKLSSGKPLVPDYAMSFIKGKSLKPFGRLWWDETVPTVVTRAEPHNQVIIHPTQARVLTIRENARLQGFPDYYRLFGPIKEKYIQVGNAVAVPVARALGYCLGQAYLGESEGSDPLYQLPPSFTSVGGRTAGQARASPVGTPAGEVVEQ

SEQ ID NO: 24 - DNA, 441 bp, Cauliflower mosaic virus
  promoter (1)..(441): CaMV 35S Promoter and flanking restriction sites
atatggatccatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctccgcggatat

SEQ ID NO: 25 - DNA, 1579 bp, Zea mays
  promoter (1)..(1579): Maize ubiquitin promoter with its intron and flanking restriction sites
atatggatccgcatgcaagctgatccactagaggccatggcggccgcactaggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtactagttccgcggatat

SEQ ID NO: 26 - DNA, 1973 bp, Artificial Sequence
  Nuclear localization signal attached to the Arabidopsis Kryptonite coding region
aattctcgagcacaccaccatgggcgatccaaagaagaagagaaaggtagaccctaagaagaagcgtaaagtcccaggcatggctggaaaaaggaaacgagctaatgctcctgaccaaacagagcgaagatcgagtgttcgggttcagaaagtgagacagaaagcgttagatgagaaggcgcgtttagtacaggagagggttaagctcctcagtgacagaaagagtgaaatttgtgtcgatgacactgagttacatgagaaagaagaggaaaatgtcgatgggagccctaaacgaagaagccctccaaagctaaccgcaatgcagaaaggaaagcagaaattgagtgtttctctgaatggtaaggacgtgaacttggaacctcatctcaaagtgacaaagtgtctgaggttatttaacaagcaatatctcctctgtgtccaggctaagttgagcaggcctgatttgaagggtgtaactgagatgataaaagctaaggcgatattgtacccaagaaaaataatcggtgaccttccaggtatagacgttggacaccgttttttttcaagagctgaaatgtgtgctgtaggattccataaccattggctaaatggcattgattatatgtcaatggaatacgaaaaagagtatagtaactacaaattaccgcttgctgtttctattgttatgtcgggccagtacgaggatgatctagacaatgcagatacagtgacttacactggacagggagggcataacttaactggtaataaacgtcagataaaggatcaacttttagaacgagggaatttggcgctaaagcactgctgcgaatataatgtgcctgtcagagtaactcgtggtcacaattgcaaaagtagctataccaaacgagtatacacttatgatggactgtacaaggttgaaaagttctgggcacaaaagggcgtttcaggatttacagtgtataagtaccgactgaaacgattggaggggcaaccagaactaactactgatcaggtcaactttgttgctggacgcataccaacgagtacttcagaaattgagggtttggtatgtgaggacatctccggagggctagaatttaagggtatccccgccactaatcgtgttgatgattcaccagtttcaccaacatctggtttcacatacatcaaatctttgattattgagcctaatgtcataattccaaagagttcaactgggtgtaactgccgaggcagctgcactgactcaaagaaatgtgcatgtgctaagcttaatgggggtaactttccatatgttgaccttaatgatggcagattaattgagtctcgagatgttgtatttgaatgtggtcctcactgtgggtgtgggccaaaatgtgtcaaccgaacttctcagaagcgtctaagattcaatcttgaggttttccgctctgcaaagaagggttgggcagttagatcatgggagtacataccagctggttcaccagtatgtgagtacataggagttgtcaggagaactgctgatgtggatactatctctgacaatgaatacatatttgagattgactgccaacagacaatgcaaggtcttggtggaagacagagaagactaagagatgttgctgtaccaatgaataatggagtcagtcagagcagtgaagatgagaatgcgccagagttctgcattgatgctggttcaacaggaaactttgctaggtttataaatcacagttgtgaaccaaacctatttgttcagtgcgtcctgagttctcaccaggatataaggcttgcccgtgtggttcttttcgcagctgacaacatttccccaatgcaggagctcacttacgactatggatatgcgcttgatagcgttcatggaccggatgggaaggtgaagcagctcgcttgctactgtggagcgctaaattgtaggaaacgcctttacccgcggaaatttggtaccatat

SEQ ID NO: 27 - protein, 20 aa, Artificial Sequence
  Synthetic Construct
MGDPKKKRKVDPKKKRKVPG

SEQ ID NO: 28 - protein, 624 aa, Artificial Sequence
  Synthetic Construct
MAGKRKRANAPDQTERRSSVRVQKVRQKALDEKARLVQERVKLLSDRKSEICVDDTELHEKEEENVDGSPKRRSPPKLTAMQKGKQKLSVSLNGKDVNLEPHLKVTKCLRLFNKQYLLCVQAKLSRPDLKGVTEMIKAKAILYPRKIIGDLPGIDVGHRFFSRAEMCAVGFHNHWLNGIDYMSMEYEKEYSNYKLPLAVSIVMSGQYEDDLDNADTVTYTGQGGHNLTGNKRQIKDQLLERGNLALKHCCEYNVPVRVTRGHNCKSSYTKRVYTYDGLYKVEKFWAQKGVSGFTVYKYRLKRLEGQPELTTDQVNFVAGRIPTSTSEIEGLVCEDISGGLEFKGIPATNRVDDSPVSPTSGFTYIKSLIIEPNVIIPKSSTGCNCRGSCTDSKKCACAKLNGGNFPYVDLNDGRLIESRDVVFECGPHCGCGPKCVNRTSQKRLRFNLEVFRSAKKGWAVRSWEYIPAGSPVCEYIGVVRRTADVDTISDNEYIFEIDCQQTMQGLGGRQRRLRDVAVPMNNGVSQSSEDENAPEFCIDAGSTGNFARFINHSCEPNLFVQCVLSSHQDIRLARVVLFAADNISPMQELTYDYGYALDSVHGPDGKVKQLACYCGALNCRKRLY

SEQ ID NO: 29 - DNA, 1973 bp, Artificial Sequence
  synthetic NLS Kryptonite
atatctcgagcacaccaccatgggcgatccaaagaagaagagaaaggtagaccctaagaagaagcgtaaagtcccaggcatggctggcaagaggaagcgcgcgaacgcccctgaccagaccgagcgcaggtcgagcgtccgcgtccagaaggtgaggcagaaggcgctggacgagaaggcgcggctggtccaggagagggtcaagctcctcagcgacaggaagagcgagatctgcgtcgacgacaccgagctgcacgagaaggaggaggaaaacgtcgacgggagccccaagcgcaggagccctcccaagctgaccgcgatgcagaaggggaagcagaagctgagcgtcagcctgaacggcaaggacgtgaacctggagccccacctcaaggtgaccaagtgcctgaggctgttcaacaagcagtacctcctctgcgtccaggccaagctgagcaggcccgacctgaagggcgtcaccgagatgatcaaggcgaaggcgatcctgtaccccaggaagatcatcggcgacctgccagggatcgacgtcggccaccgcttcttcagcagggccgagatgtgcgccgtcggcttccacaaccactggctgaacggcatcgactacatgagcatggagtacgagaaggagtacagcaactacaagctgccgctggcggtgagcatcgtcatgagcggccagtacgaggacgacctggacaacgcggacaccgtgacctacaccgggcagggagggcacaacctgaccggcaacaagcgccagatcaaggaccagctgctggagcgcgggaacctggcgctgaagcactgctgcgagtacaacgtgcccgtcagggtcacccggggtcacaactgcaagagcagctacaccaagcgcgtgtacacctacgacgggctgtacaaggtcgagaagttctgggcgcagaagggcgtgagcgggttcaccgtgtacaagtaccggctgaagcggctggaggggcagcccgagctgaccaccgaccaggtcaacttcgtcgccggacgcatcccgaccagcaccagcgagatcgaggggctggtgtgcgaggacatcagcggagggctggagttcaaggggatccccgccaccaaccgcgtggacgacagcccggtcagcccgaccagcgggttcacctacatcaagagcctgatcatcgagcccaacgtcatcatccccaagagcagcaccgggtgcaactgccgcggcagctgcaccgacagcaagaagtgcgcgtgcgcgaagctgaacgggggtaacttcccctacgtggacctgaacgacggcaggctgatcgagagccgggacgtggtcttcgagtgcgggccgcactgcgggtgcgggccgaagtgcgtcaaccggacctctcagaagcggctgaggttcaacctggaggtgttccgcagcgcaaagaaggggtgggcggtcaggagctgggagtacatccccgctgggagccccgtgtgcgagtacatcggggtggtcaggaggaccgcggacgtggacaccatcagcgacaacgagtacatcttcgagatcgactgccagcagaccatgcaggggctgggcgggaggcagaggaggctgagggacgtcgcggtccccatgaacaacggggtcagccagagcagtgaggacgagaacgcgcccgagttctgcatcgacgcggggagcaccgggaacttcgcgaggttcatcaaccacagctgcgagcccaacctgttcgtccagtgcgtcctgagcagccaccaggacatcaggctggcccgcgtggtcctgttcgcggcggacaacatcagccccatgcaggagctcacctacgactacgggtacgcgctggacagcgtccacgggccggacgggaaggtgaagcagctcgcctgctactgcggggcgctgaactgcaggaagcgcctgtacccgcggaaatttggtaccatat

SEQ ID NO: 30 - protein, 20 aa, Artificial Sequence
  Synthetic Construct
MGDPKKKRKVDPKKKRKVPG

SEQ ID NO: 31 - protein, 624 aa, Artificial Sequence; identical to SEQ ID NO: 28
  Synthetic Construct